(19) United States
(12) Patent Application Publication
(10) Pub. No.: US 2006/0135921 A1
(43) Pub. Date: Jun. 22, 2006

(54) Porous particulate collagen sponges

(76) Inventors: Robert A. Wiercinski, Lincoln, MA (US); Roy H. L. Pang, Etna, NH (US); Dona Hevroni, Lexington, MA (US)

Correspondence Address: William L Baker;W R Grace & Company Conn, Patent Department, 62 Whittemore Avenue, Cambridge, MA, 02140-1692, US

(21) Appl. No.: 10/552,038

(22) PCT Filed: Apr. 5, 2004
(86) PCT No.: PCT/US04/10564

§ 371 Date: Nov. 18, 2005

(51) Int. Cl. B32B 3/26; A61F 13/15

(52) U.S. Cl. 604/368; 428/304.4

(57) ABSTRACT

The present invention relates to the development of new porous particulate collagen sponges, combining the desirable features of low toxicity, resorbability, and satisfactory porosity, particularly when wetted in an aqueous medium. Accordingly, the present invention is directed to new porous, particulate, dehydrothermally cross-linked, wetted sponges, as well as a process for making them.

### RELATED APPLICATIONS

This application claims priority to U.S. Provisional Application 60/460,341, filed on Apr. 4, 2003, entitled “Porous Particulate Collagen Sponges” and U.S. Provisional Application 60/513,922, filed on Oct. 23, 2003, entitled “Porous Particulate Collagen Sponges.” This application is related to U.S. Provisional Application 60/370,043, filed on Apr. 4, 2002, entitled “Tissue Composites and Uses Thereof”; and PCT Application PCT/US03/10439 filed on Apr. 4, 2003, entitled “Tissue Composites and Uses Thereof” The contents of all of the above-referenced applications are hereby incorporated herein by reference in their entireties.

### BACKGROUND OF THE INVENTION

Injuries to soft tissues are extremely common in hospital clinics. In fact, soft tissue replacements amount to an estimated 35% of the world market for all medical devices (Materials Technology Foresight in Biomaterials, Institute of Materials, London (1995).

There have been many options proposed for the repair of soft tissues. These generally involve synthetic materials, biological materials or a combination of the two. Synthetic alternatives have demonstrated in vivo instability, and thus relatively poor long-term performance. Biological solutions traditionally involve autografts, allografts or xenografts, depending on the source of tissues. Each of these options has proved to be far from ideal with, for example, autografts leading to donor site morbidity, and allografts and xenografts to graft rejection.

The relative failure of many surgical, synthetic, and graft solutions has led to a growing interest in the development of cell-seeded or tissue-engineered repair systems to address a number of clinical problems related to tissues, e.g., connective tissue or soft tissue. Such repair systems typically involve autologous or allogenic cells that are isolated from a tissue biopsy at a site remote to the injury. Typically, the isolated cells are expanded in cell culture and seeded in a suitable three-dimensional scaffold material, which when implanted into the injured site elicits a biological repair.

While previous studies have examined collagen sponges or foams for use as hemostatic agents, more recent attempts have examined collagen scaffolds for tissue repair in vivo, and as research tools in vitro for seeding various cell types in the study of cell function in three dimension (see e.g., U.S. Pat. No. 5,109,934). As collagen sponges have a low immunogenicity, and consist of a naturally occurring structural protein, cells can attach, interact with and degrade scaffolds of this type.

Collagen is a preferred material for tissue engineering because the extracellular matrix of animal tissue comprises a sponge-like collagen network. However, it has been difficult to create a man-made, sponge-like collagen network, from purified insoluble or soluble collagen obtained from an animal source that mimics the natural extracellular matrix. Man-made sponges in various forms, including sheets and particulates are known, but have not exhibited the most desirable combination of properties, e.g., resorbability, no toxicity, and satisfactory porosity, particularly when wetted in an aqueous medium.

In fact, known man-made sponges are usually cross-linked to provide the degree of wet strength and measured resistance to dissolution needed for therapeutic efficiency. Cross-liking of the sponges may be induced chemically, thermally (e.g., dehydrothermal cross-linking), or by radiation, e.g., ultraviolet or gamma radiation. Cross-linking agents for known for their use in chemical cross-linking include but are not limited to glutaraldehyde, formaldehyde and like aldehydes; hexamethylene diisocyanate, tolylene diisocyanate, and like diisocyanates; ethyleneglycol diglycidylether, and like epoxides; and carbodiimide hydrochlorides.

Using typical chemical cross-linking agents, like gluteraldehyde, to prepare collagen sponges it is possible to tailor formulations such that the sponge can be wetted directly into an aqueous medium without collapsing the porous structure. However, such agents are toxic, and sponges cross-linked with external agents may not be easily resorbable. In addition, while collagen sponges are known that have been dehydrothermally cross-linked to overcome problems with toxicity and resorbability, both the large pore size and shrinkage/reduction of porosity that occurs upon wetting directly in an aqueous medium have not reached the sought after paradigm in tissue engineering.

### SUMMARY OF INVENTION

The objective of the present invention is the development of new porous particulate collagen sponges, combining the desirable features of low toxicity, resorbability, and satisfactory porosity, particularly when wetted in an aqueous medium. Accordingly, the present invention is directed to new porous, particulate, dehydrothermally cross-linked, wetted sponges, as well as a process for making them.

Accordingly, one aspect of the invention is directed to a dehydrothermally, cross-linked collagen sponge wetted with an aqueous medium wherein the structure of the wetted sponge is substantially retained.

In another aspect, the invention is a dehydrothermally cross-linked, collagen sponge wetted with an aqueous medium prepared by a method comprising:
(a) preparing an aqueous dispersion of insoluble collagen or solution of soluble collagen;
(b) casting the dispersion or the solution into a shape desired for end use;
(c) freezing the cast shape;
(d) lyophilizing the frozen, cast shape to form a collagen sponge;
(e) dehydrothermally cross-linking the lyophilized collagen sponge;
(f) wetting the dehydrothermally cross-linked sponge in a non-aqueous water soluble solvent; and
(g) washing the sponge wetted with a non-aqueous-water soluble solvent with an aqueous solution.

Another aspect of the invention is a dehydrothermally cross-linked, collagen sponge wetted with an aqueous medium prepared by a method comprising:
(a) preparing a dehydrothermally cross-linked collagen sponge;
(b) wetting the dehydrothermally cross-linked sponges in a non-aqueous water soluble solvent at reduced pressure, resulting in dehydrothermally cross linked sponge wetted with a non-aqueous medium; and
(c) exposing the wetted, dehydrothermally cross-linked sponge to a gradient of solvent mixtures comprising the non-aqueous solvent and water, starting with a high concentration of the non-aqueous solvent and ending with water or an aqueous solution to form a dehydrothermally cross-linked sponge wetted with an aqueous medium.

In an additional aspect, the present invention is directed to a dehydrothermally cross-linked, collagen sponge wetted with an aqueous medium prepared by a method comprising:
(a) preparing a dehydrothermally cross-linked collagen sponge;
(b) wetting the dehydrothermally cross-linked sponge in a non-aqueous water soluble solvent at reduced pressure, resulting in a dehydrothermally cross-linked sponge wetted with a non-aqueous medium; and
(c) washing or wetting with an aqueous medium.

A further aspect of the invention is a particulate, dehydrothermally cross-linked, collagen sponge wetted with an aqueous medium prepared by a method comprising:
(a) preparing an aqueous dispersion of insoluble collagen or solution of soluble collagen;
(b) casting the dispersion or the solution into a shape;
(c) freezing the cast shape;
(d) milling the shape into particles at a temperature below the freezing point of the particles;
(e) lyophilizing the frozen particles to form collagen sponge;
(f) dehydrothermally cross-linking the lyophilized collagen sponge;
(g) wetting the dehydrothermally cross-linked sponge in a non-aqueous water soluble solvent at reduced pressure, resulting in dehydrothermally cross-linked sponges wetted with a non-aqueous medium; and
(h) exposing the wetted, dehydrothermally cross-linked sponge to a gradient of solvent mixtures comprising the non-aqueous solvent and water, starting with a high concentration of the non-aqueous solvent and ending with water or an aqueous solution to form a dehydrothermally cross-linked sponges wetted with an aqueous medium.

In yet another aspect, the present invention relates to a particulate, dehydrothermally cross-linked, collagen sponge wetted with an aqueous medium prepared by a method comprising:
(a) preparing an aqueous dispersion of insoluble collagen or solution of soluble collagen;
(b) casting the dispersion or the solution into a shape;
(c) freezing the cast shape;
(d) milling the shape into particles at a temperature below the freezing point of the particles in a coolant medium;
(e) separating the milled particles into ranges by sieving in the coolant medium;
(f) lyophilizing the frozen particles to form collagen sponges;
(g) dehydrothermally cross-linking the lyophilized collagen sponges;
(h) wetting the dehydrothermally cross-inked sponges in a non-aqueous water soluble solvent at reduced pressure, resulting in dehydrothermally cross-linked sponges wetted with a non-aqueous medium; and
(i) exposing the wetted, dehydrothermally cross-linked sponges to a gradient of solvent mixtures comprising the non-aqueous solvent and water, starting with a high concentration of the non-aqueous solvent and ending with water or an aqueous solution to form a dehydrothermally cross-linked sponges wetted with an aqueous medium.

In yet another aspect, the invention is a particulate, dehydrothermally cross-linked, collagen sponge wetted with an aqueous medium prepared by a method comprising:
(a) preparing an aqueous dispersion of insoluble collagen or solution of soluble collagen;
(b) casting the dispersion or the solution into a shape;
(c) freezing the cast shape;
(d) milling and sieving the shape into particles simultaneously at a temperature below the freezing point of the particles in a coolant medium lyophilizing the frozen particles to form collagen sponges;
(e) lyophilizing the frozen particles to form collagen sponges;
(f) dehydrothermally cross-linking the lyophilized collagen sponges;
(g) wetting the dehydrothermally cross-linked sponges in a non-aqueous water soluble solvent at reduced pressure, resulting in dehydrothermally cross-linked sponges wetted with a non-aqueous medium; and
(h) exposing the wetted, dehydrothermally cross-linked sponges to a gradient of solvent mixtures comprising the non-aqueous solvent and water, starting with a high concentration of the non-aqueous solvent and ending with water or an aqueous solution to form a dehydrothermally cross-linked sponges wetted with an aqueous medium.

An additional aspect of the invention is a particulate, man-made, non-spherical, dehydrothermally cross-linked, collagen sponge.

In another aspect, the present invention is directed to a particulate, man-made, non-spherical, dehydrothermally cross-linked, wetted collagen sponge.

In yet another aspect, the invention is a particulate, non-spherical dehydrothermally cross-linked, collagen sponge prepared by a method comprising:
(a) preparing an aqueous dispersion of insoluble collagen or solution of soluble collagen;
(b) casting the dispersion or the solution into a shape;
(c) freezing the cast shape;
(d) milling the shape into, particles at a temperature below the freezing point of the particles in a coolant medium;
(e) separating the milled particles into ranges by sieving in the coolant medium;
(f) lyophilizing the frozen particles to form collagen sponges; and
(g) dehydrothermally cross-linking the lyophilized collagen sponges.

An additional aspect of the present invention is a particulate, non-spherical dehydrothermally cross-linked, collagen sponge prepared by a method comprising:
(a) preparing an aqueous dispersion of insoluble collagen or solution of soluble collagen;
(b) casting the dispersion or the solution into a shape;
(c) freezing the cast shape;
(d) milling and sieving the shape into particles simultaneously at a temperature below the freezing point of the particles in a coolant medium;
(e) lyophilizing the frozen particles to form collagen sponges; and
(f) dehydrothermally cross-linking the lyophilized collagen sponges.

In another aspect, the present invention is a population of non-spherical, dehydrothermally cross-linked, collagen sponges wetted with an aqueous medium wherein the average cross-sectional area or max. diameter of wetted sponges are within ±20% of the value for the average cross-sectional area or max. diameter for the unwetted sponge.

Additionally, one aspect of the present invention is directed to a spherical, dehydrothermally cross-linked, collagen sponge wherein the average maximum diameter of the pores on the surface of the particle is 2.5μ to 5μ.

In another aspect, the invention is a spherical, dehydrothermally cross-linked, collagen sponge wherein the average maximum diameter of the pores on the surface of the particle is 3μ to 5 μ.

In yet another aspect, the invention is directed to a spherical, dehydrothermally cross-linked collagen sponge, wherein the average area of the pores on the surface of the particle is >4 mm².

A further aspect of the invention is directed to a spherical, dehydrothermally cross-linked, collagen sponge wherein ≧30% of the surface pore area is occupied by pores that have a maximum diameter of ≧10 microns.

Another aspect of the invention pertains to a population of spherical, dehydrothermally cross-linked, collagen sponges wetted with an aqueous medium.

Yet another aspect of present invention is a population of spherical, dehydrothermally cross-linked, collagen sponges wetted with an aqueous medium wherein the structure of the wetted sponge is substantially retained.

In another aspect, the invention pertains to a process for wetting a sponge with an aqueous medium comprising wetting a sponge with a sequence of five wetting agents, wherein the sequence of five wetting agents comprises:

100% to 95% non-aqueous, water soluble solvent/0% to 5% water;

94% to 65% non-aqueous, water soluble solvent/6% to 35% water;

64% to 35% non-aqueous, water soluble solvent 36% to 65% water;

34% to 6% non-aqueous, water soluble solvent/66% to 94% water; and

0% to 5% non-aqueous, water soluble solvent/600% to 95% water.

In yet another aspect, the present invention pertains to a process for wetting a sponge with an aqueous medium comprising wetting a sponge with a sequence of four wetting agents, wherein the sequence of four wetting agents comprises:

100% to 95% non-aqueous, water soluble solvent/0% to 5% water;

94% to 50% non-aqueous, water soluble solvent/6% to 50% water;

49% to 6% non-aqueous, water soluble solvent/51% to 94% water; and

0% to 5% non-aqueous, water soluble solvent/100% to 95%.

An additional aspect of the invention is directed to a process for wetting a sponge with an aqueous medium comprising wetting a sponge with a sequence of two wetting agents, wherein the sequence of two wetting agents comprises:

100% to 95% non-aqueous, soluble solvent; and water.

Another aspect of the present invention is a carrier device comprising wetted spherical and/or non-spherical particulates, of the present invention, and a microorganism.

In an additional aspect, the invention pertains to a carrier device comprising the wetted spherical and/or non-spherical particulates, of the present invention, and cells.

In yet an additional aspect; the invention is a composite comprising the spherical and/or non-spherical particulates and a pharmaceutical agent.

A further aspect of the present invention pertains to a process for preparing a carrier device coated with a complex coacervate comprising adding the carrier device of claim 64 or 67 to a solution comprising a component of a complex coacervate, wherein the carrier device further comprises a second component of the complex coacervate.

Another aspect of the invention is directed to a continuous process for preparing sheet-like single layer and multiple layer engineered tissue matrices comprising cells, a particulate biopolymer scaffold, and a biopolymer gel comprising the following steps:
(a) mixing an aqueous dispersion of a particulate biopolymer scaffold with cells dispersed in a solution of a gellable biopolymer at a temperature at which the gellable biopolymer solution will not gel;
(b) casting the mixture of cells, particulate biopolymer scaffold, and biopolymer gel onto a film in a continuous web process; and
(c) heating the mixture to a temperature at which the gellable biopolymer solution gels.

Yet another aspect of the invention pertains to a process for producing multiple layer matrices comprising
preparing a first layer prepared by the process of claims 76, 77, or 78; and
casting a second layer onto the first layer, wherein the second layer is prepared by the process of claims 76, 77, or 78; wherein the second layer comprises cells dispersed in a biopolymer gel; or wherein the second layer comprises an aqueous dispersion of cells,

wherein the second layer is cast onto the first layer in a continuous web process.

Yet another aspect of the invention is a composite produced from the process described in Example 17B, with or without the porous film.

### BRIEF DESCRIPTION OF THE DRAWINGS

FIG. 1 is a series of confocal microscopy images depicting wetted particulates of the invention and illustrating the comparison of the porosity for samples wetted via the nine step process to that for samples wetted directly in PBS.

FIG. 2 is a series of SEM images depicting the dry particulates of the invention (as shown in FIG. 1) and illustrating the comparison of the porosity/particulate structure for the dry samples using different casting methods.

FIG. 3 is a schematic representation of the apparatus used to grind and separate frozen cubes of biopolymer sponges in the preparation of non-spherical particles of the invention.

FIG. 4 is a series of visible light microscopy images depicting dry and wet particulate sponges (Sample No. 7 of the Overview of the Invention) after a two-step wetting process and a direct wetting process, illustrating the significant difference in structure retention between the two methods.

FIG. 5 is a series of visible light microscopy images of a sheet sponge, frozen at −20 C, wetted via the two step process, direct wetting in 70% alcohol, and direct wetting in PBS, illustrating the effects of the different wetting procedures on sheet sponges.

FIGS. 6A-C are confocal microscopy images depicting the proliferation of porcine fibroblasts in three types of particles to illustrate differences in the cell density of cultures in FIGS. 6B and 6C that were incubated for 10 days in spinner flasks when compared to FIG. 6A that was incubated for 6 days in a 100 mm Petri dish.

FIG. 7 is a bar graph representing the pore area percentage as a function of pore diameter for sample that was frozen in liquid nitrogen (Sample No. 1).

FIG. 8 is a bar graph representing the pore area percentage as a function of pore diameter for sample that was frozen in pentane at −15 C (Sample No. 4).

FIG. 9 is a bar graph representing the percentage the total particle area as a function of the particle size, and illustrates the particle size distribution of non-spherical particles.

FIG. 10 is a set of confocal microscopy images depicting porcine fibroblasts cultured in vitro on porous collagen spheres that were cast using pentane.

FIG. 11 is a schematic of the apparatus used in the continuous process for producing sheet-like matrices (Example 17A).

FIG. 12 is a schematic representation of a biocompatible porous particulate scaffold in contact with a biocompatible gel seeded with cells, wherein the gel and scaffold are layered on porous film. This is a schematic representation of the tissue matrix generated using the apparatus shown in FIG. 11 (Example 17A).

FIG. 13 is a schematic representation of a biocompatible porous scaffold filled with a nutrient solution and seeded with cells, in contact with a nonporous biocompatible gel, wherein the gel and scaffold are layered on porous film. This is a schematic representation of a tissue matrix generated using the apparatus shown in FIG. 11 (Example 17B).

FIG. 14 is a schematic of the apparatus used in the continuous process for producing a two layer tissue matrix comprising sheet-like matrices (Example 17C). The apparatus is similar to that shown in FIG. 11 with the exception that it contains two coating stations.

FIG. 15 is a schematic representation of a multicellular composite prepared by the apparatus of FIG. 14 (Example 17C).

FIG. 16 is an illustration of the line placement and line measurement superimposed on a confocal microscopy image of a wetted particle as performed using the methodology of Example 11.

### DETAILED DESCRIPTION OF INVENTION

The present invention is directed to the development of sponges where sponge size, sponge shape, and pore size are maintained when the dry sponges, e.g., particulates and sheets, are wetted with an aqueous medium.

Accordingly, in one embodiment, the present invention is directed to a dehydrothermally, cross-linked sponge wetted with an aqueous medium wherein the structure of the wetted sponge is substantially retained. Additionally, the invention is directed to methods of preparation of these sponges and methods of use thereof.

The term “sponge” as used herein, is synonymous with the term “scaffold,” and includes materials that provide a support structure, e.g., for cells or in-growth of cells, and are suitable for containing a biological material, e.g., a biological solution. The sponges of the present invention include non-spherical particulate, spherical particulate, and non-particulate sponges, e.g., sheet sponges, prepared by the methods described herein.

The sponge may comprise any biocompatible material, preferably a porous material, such as a porous biopolymer. Examples of commercially available biocompatible materials include collagen, e.g., types I to XXI including -I, -II, -III, and -IV, gelatin, alginate, agarose, e.g., type-VII, carrageenans, glycosaminoglycans, proteoglycans, polyethylene oxide, poly-L-lactic acid, poly-glycolic acid, polycaprolactone, polyhydroxybutarate, polyanhydrides, fibronectin, laminin, hyalironic acid, chitin, chitosan, EHS mouse tumor solubilized extract, and copolymers of the above. However, the specific use of non-resorbable polymeric components, or of non-polymeric resorbable components such as soluble bioglasses is not precluded.

In a preferred embodiment, the sponge comprises collagen, including any one or combination of the 21 types of the known collagen types, e.g. types -I, -II, -III, -IV, etc. In one particular embodiment; the collagen is insoluble collagen. In another embodiment, soluble collagen may also be used. In a specific embodiment of the invention, the biopolymer is a cross-linked collagen, for example, bovine Type I collagen.

Collagen for use in the sponges of the invention is commercially available, for example, from Sigma Aldrich in a variety of forms. Collagen useful in the present invention may be derived from human, as well as animal sources. Moreover, such collagen may be extracted from animal tissue, e.g., bovine or porcine tissues, e.g., as described by Bell et al. in U.S. Pat. No. 5,709,934. Recombinantly produced human and animal collagens, which are produced by a synthetic process by Fibrogen, may also be used in the methods of the present invention.

The biopolymer sponges, e.g., collagen sponges, may be thermally cross-linked (e.g., dehydrothermal cross-linking). In fact, in particular embodiments of the invention, the present invention does not use toxic cross-linking agents, e.g., chemical cross-linking agents, like glutaraldehyde. In certain embodiments, the present invention does not utilize chemical modification.

In one embodiment, a dehydrothermally cross-linked, collagen sponge wetted with an aqueous medium may be prepared by a method comprising:
(a) preparing an aqueous dispersion of insoluble collagen or solution of soluble collagen;
(b) casting the dispersion or the solution into a shape desired for end use;
(c) freezing the cast shape;
(d) lyophilizing the frozen, cast shape to form a collagen sponge;
(e) dehydrothermally cross-linking the lyophilized collagen sponge;

The sponges of the present invention may contain optional ingredients that may be added to the collagen dispersion or collagen solution prior to casting and freezing, including proteins, carbohydrates, and lipids. However, the methods of preparation described herein typically involve the preparation of a dispersion comprising at least a 0.05% to 10.0% dispersion of insoluble or soluble collagen, e.g., 0.1% to 1.0%, e.g., a 0.3% to 0.7%. Such dispersions also comprise comprises 1% to 20% glacial acetic acid, e.g., 1% to 5%.

The dispersion is subsequently cast, frozen, lyophilized and then dehydrothermally cross-linked at elevated temperatures, e.g., at a temperature between 80 C and 150 C, and at decreased pressures, e.g., at a pressure of less than 5 torr, e.g., less than 1 torr.

In one embodiment of the present invention, the dehydrothermally cross-linked sponges are wetted using a non-aqueous water soluble solvent, followed by washing the sponges with an aqueous solution. In certain embodiments, the washing step involves washing the sponges with a series of non-aqueous water soluble solvent/water mixtures starting with a mixture comprising a high level of the non-aqueous water soluble solvent and then stepwise with mixtures comprising progressively higher levels of water.

A preferred process for producing dry sponges involves preparing a dispersion or solution of collagen in an aqueous, acidic medium, casting the aqueous mixture into the desired shape, freezing in a coolant medium, and then lyophilizing. In certain embodiments, the casting process involves pumping the dispersion or solution through a narrow tube into air, or involves casting a shape in a mold. Moreover, the freezing process may utilize a freezing medium of air, a gas, liquid nitrogen, a cryogenic liquid, or a water-insoluble organic solvent.

The pore size of the dry sponge depends upon freezing conditions, collagen concentration, and pH. More than other variables, freezing conditions affect the pore size of dry collagen sponges. In this regard, and without wishing to be bound by theory, pore size depends on the size of the ice crystals formed in the freezing step, and the size of the crystals is indirectly proportional to the freezing rate. If freezing is performed isothermally in a liquid medium, pore size is proportional to the temperature of the freezing medium.

Freezing in liquid nitrogen results in very small pores e.g., of about 5μ to 10μ (for the largest pores). Freezing in liquid pentane, at about −15 C, results in larger pores of up to about 40μ to 50μ. Much higher temperatures can not be used as the aqueous mixture will not freeze. However, if larger pores are desired, a gas may be used as the freezing medium (i.e., at fixed temperature, heat transfer and freezing rate are slower in a gas than a liquid). Moreover, freezing in air at about −20 C results in pores sizes up to about 200 μ.

Utilizing the methods presented herein, wetted particulates, e.g., spherical and non-spherical, of varying shape, particle size, and pore size have been produced. Smaller particles with smaller pores may be prepared from the methods of the invention. Particle sizes as low a 1μ may be made by the methods of the invention. Large spherical and non-spherical particle may also be made, e.g., particles as big as 10,000μ, or even larger may be made by the methods of the invention.

In a specific embodiment, the spherical particulates of the present invention were produced by freezing in a liquid media and the largest pore sizes ranged from 5μ to 50μ. Although, it is contemplated by the invention to freeze spherical particulates in a gas at an appropriate temperature to yield larger pores. Spherical particles of 1μ to 10,000μ, e.g., 250μ to 2000μ, e.g., 500μ to 2000μ, in diameter with wet maximum pore sizes from 5μ to 50μ were produced. A spherical, dehydrothermally cross-linked, wetted or unwetted collagen sponge (or a population of sponges) may be prepared by the processes of the invention, with the ability to tailor the properties of the sponges. For example, the average maximum diameter of the pores on the surface of the particle, e.g., prepared by casting in pentane, may be about 2.5μ to about 5 μ, e.g., about 3μ to about 5μ; the average area of the pores on the surface of the particle may be >4 mm²; the sponge diameter is 0.25 to 10 mm, e.g., 0.5 to 3 mm; and ≧30% of the surface pore area is occupied by pores that have a maximum diameter of ≧10 microns.

Non-spherical particles of 0.5 to 4 mm in diameter (and larger) and dry and wet maximum pore sizes from 5μ, to 200μ were also produced. A spherical, particulate, man-made, non-spherical, dehydrothermally cross-linked, wetted or unwetted collagen sponge (or a population of sponges may be prepared by the processes of the invention, with the ability to tailor the properties of the sponges. For example, 50% of the total cross-sectional area of population of sponges may be made up by particles with a diameter ranging from 1 to 2.5 mm; the average roundness may be 22, the average max pore diameter may be 3μ to 16μ; the average pore area may be 10 to 200 mm²; the average max. particle diameter may be 0.5 to 10 mm; and the average max. particle diameter may be 0.1 to 25 mm.

Non spherical particles are produced in the processes where freezing may be done in a liquid or gas medium. Non-spherical particulates with maximum pore sizes ranging from 5μ to 200μ were produced. Non spherical particulates are produced by preparing a dispersion or solution, casting into a shape that is much larger than the size of the desired particulate, freezing, milling, and lyophilization. In this regard, a cryogenic milling process can be utilized. Furthermore, particle size may be controlled by fractioning the frozen, ground dispersion, with a series of sieves in, for example, liquid nitrogen. However, other chilled liquids that would be useful for freezing the particulate may also be used as the grinding and separation medium.

In a particular embodiment of the invention, three fractions of non-spherical particulate sizes are produced including one passing through a 5 mm sieve and retained on a 2 mm sieve, a 2^{nd} passing through a 2 mm sieve and retained on a 0.5 mm sieve, and the third fraction is the remainder. However, other particle sizes are contemplated by the invention, and would be determined by the sieves utilized. In certain embodiments, the cryogenic milling process and the separation of the particle sizes through the use of one or more sieves may be performed simultaneously. Advantageously, higher yields of the desired particle fractions may be produced in comparison to the process that utilizes separate grinding and sieving steps.

Smaller dry particulates and processes to manufacture smaller dry particulates are also contemplated. One option is to spray a solution or a dispersion directly into a liquid freezing bath. Another option is producing a “water in oil” emulsion, wherein the “water phase” is the solution or dispersion. The temperature of the emulsion is maintained below the freezing point of the dispersion or the solution. The frozen particulates made by either process are then lyophilized to produce the dry particulate sponges.

Retention of Structure Upon Wetting of Collagen Sponges:

In general, the lyophilized, dehydrothermally, cross-linked, sponges, e.g., known sponges as well as sponges of the present invention, can be directly wetted with, aqueous medium, e.g., buffer, or a biological solution, e.g., a nutrient solution. However this causes shrinkage and reduction of pore size for dehydrothermally cross-linked sponges. More specifically, the wetting process of the invention is intended to be useful for all sponges, regardless of their method of preparation. For example, in addition to preparation by the methods of the present invention, sponges that may benefit from the wetting processes described herein may be prepared from solutions that are directly dehydrated using heat and or vacuum to produce the sponge morphology, which may then be dehydrothermally cross-linked.

The term “structure” as used herein is defined as the quantitative and qualitative physical structure of the particulate, e.g., spherical or non-spherical; or sheet sponge material, including relative porosity, cross-sectional area, maximum diameter of the pores, and maximum diameter of the sponge,

The language “substantially retained” as used herein, refers to the retention of the the structural attributes of a sponge (or a population of sponges) of the present invention upon wetting with an aqueous medium. For example, upon wetting with an aqueous medium, the porosity, e.g., pore shape and size, as well as the relative porosity of the sponges is maintained; the volume, cross-sectional area, and/or maximum diameter of the wetted sponge is retained, within ±20%, e.g., within ±10%, e.g., within ±5%, of the value for volume, cross-sectional area, and/or maximum diameter of the unwetted sponge.

In certain embodiments, the collagen sponges of the present invention are man-made or non-naturally occurring. This is distinguished from a naturally-occurring sponge from a human or animal source. Natural tissue comprises a collagen sponge and cells. A naturally occurring sponge is produced by de-cellularization of natural tissue leaving the collagen sponge, which retains the natural sponge-like properties. For the man-made sponges, the source of collagen may be animal or human, but the naturally occurring sponge is first reduced to an insoluble fiber or powder or a soluble solution of collagen. It is then reconstructed into a man made sponge.

In certain embodiments, the morphology of sponges of the present invention is unique. In one embodiment the distinction in the morphology of the sponges is the result of the source of the collagen used to prepare the sponge, e.g., the collagen is commercially processed beyond the point that permits retention of natural sponge-like properties (e.g., there is a loss of natural morphology), as opposed to derived directly from natural sources that allow retention of the natural sponge-like properties.

It should be noted that both the process of preparation of the wetted sponges and the sponges prepared from the wetting process, including further preparations that use the wetted sponges, e.g., composites, described herein are contemplated by the present invention. For, example, one embodiment of the invention is a process for wetting sponges with a sequence of five wetting agents and the sequence of five wetting agents comprises:

100% to 95% non-aqueous, water soluble solvent/0% to 5% water;

94% to 65% non-aqueous, water soluble solvent/6% to 35% water;

64% to 35% non-aqueous, water soluble solvent/36% to 65% water;

34% to 6% non-aqueous, water soluble solvent/66% to 94% water; and

0% to 5% non-aqueous, water soluble solvent/100% to 95% water, as well as the wetted sponges and composites made therefrom.

In certain embodiments, the non-aqueous solvent is ethanol, isopropanol, methanol, acetone, dimethyl ether, other water soluble alcohols and ketones. In a specific embodiment, the non-aqueous solvent is ethanol.

In an additional embodiment, the invention is directed to a process for wetting sponges with a sequence of four wetting agents and the sequence of four wetting agents comprises

100% to 95% non-aqueous, water soluble solvent/0% to 5% water;

94% to 50% non-aqueous, water soluble solvent/6% to 50% water;

49% to 6% non-aqueous, water soluble solvent/51% to 94% water; and

0% to 5% non-aqueous, water soluble solvent/100% to 95%, as well as the wetted sponges and composites made therefrom.

Another embodiment of the invention is a process for wetting sponges with a sequence of two wetting agents and the sequence of two wetting agents comprises: 100% to 95% non-aqueous, soluble solvent; and water, as well as the wetted sponges and composites made therefrom.

The language “biological solution” as used herein is defined as a biological material, e.g., cells, contained in a liquid medium, e.g., aqueous solutions, e.g., water or buffered aqueous solutions. In one embodiment, the biological solution is a nutrient solution supportive of cell growth. However, it should also be noted that the biological material may be the liquid medium, for example, water or buffered solutions.

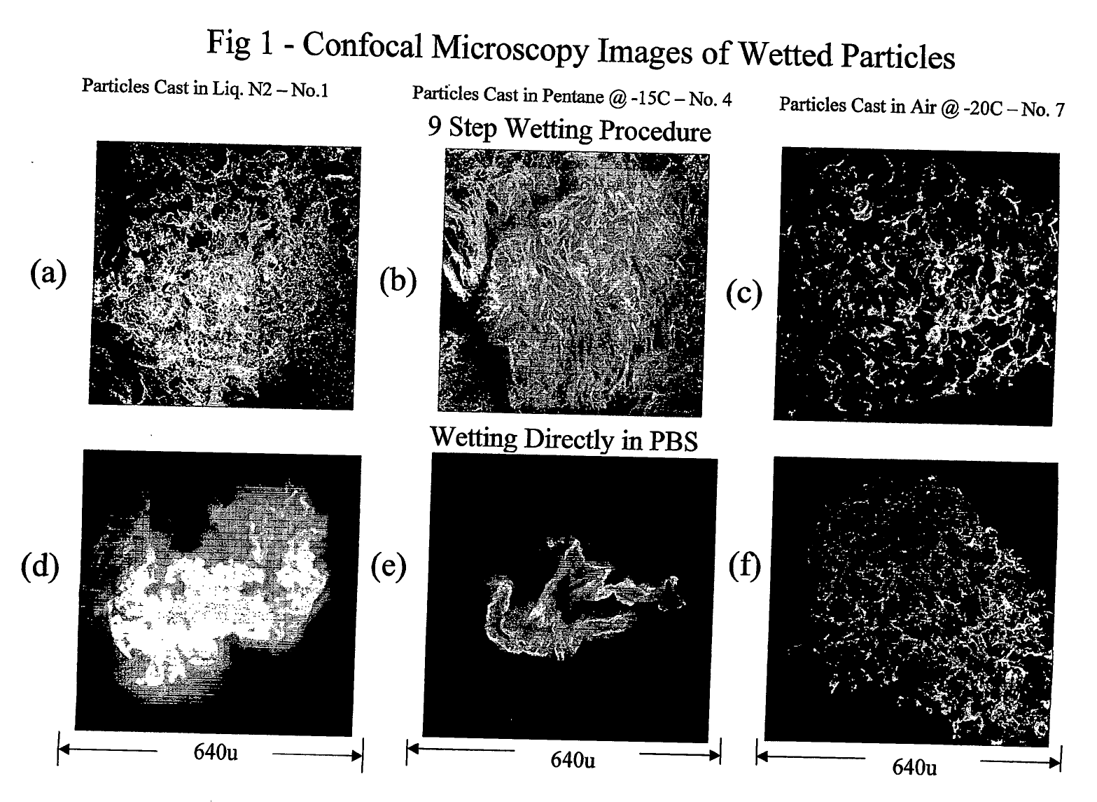

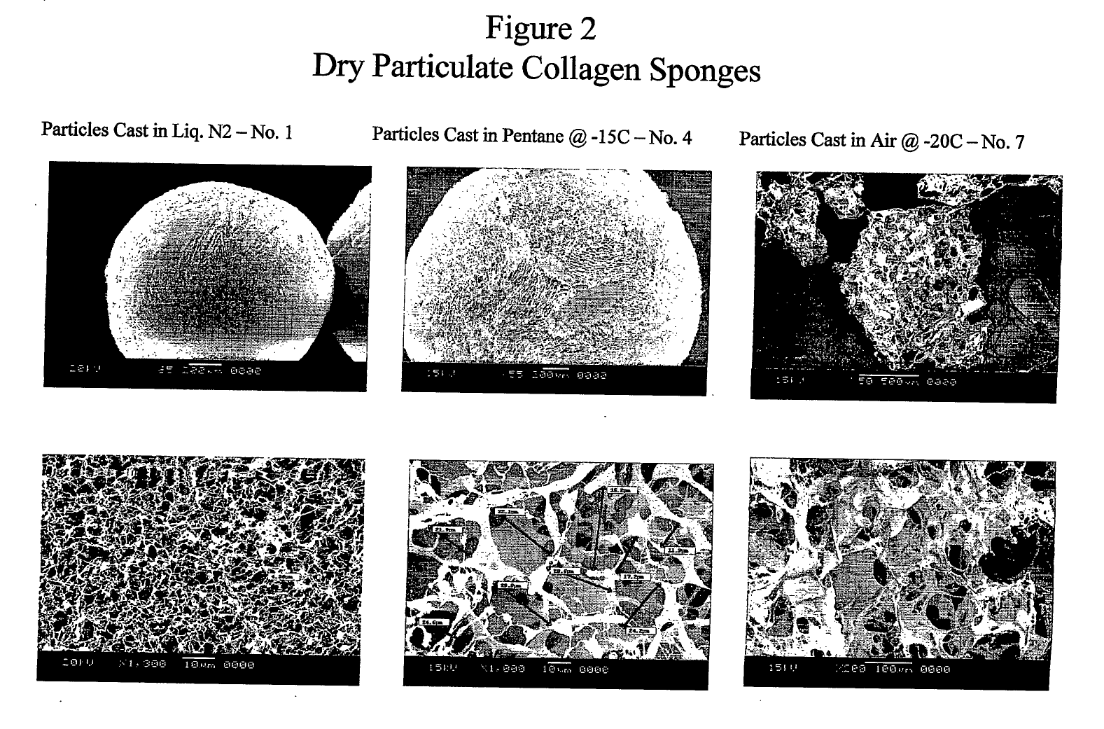

In one embodiment, the invention is directed to a stepwise method for the retention of porosity upon wetting a dehydrothermally cross-linked collagen sponge with an aqueous medium. This can be best appreciated from an examination of the confocal microscopy images in FIG. 1 for sample nos. 1, 4, and 7 described in the Overview of the Exemplification. Comparison of the porosity for samples wetted via the nine step process (described in Example 3 and the Overview of the Exemplification) may easily be made to that for samples wetted directly in PBS. Samples 1 and 4 are porous when wetted via the nine step process, and pore size is similar to that for the dry samples in FIG. 2. When samples 1 and 4 are wetted directly in PBS, the porosity is totally collapsed. Sample 7 comprises much larger pores than samples 1 and 4. Sample 7 is porous when wetted via the nine step process and porosity is similar to that for the dry sample. When sample 7 is wetted directly in PBS there is some collapse of porosity versus the nine step method, but the reduction in porosity is not as dramatic as that for the smaller pore size samples, 1 and 4.

Particle size measurements complement confocal microscopy results. Moreover, reduction of particle size upon wetting is an indirect method of measuring reduction of porosity. The order of porosity reduction upon wetting, as measured via change in particle diameter or particle cross-sectional area is as follows: direct in PBS (32% to 67%)>direct in 70% ethanol (41%)>>2 step (−0.2% to 17.7%) 2 step process (−1.6% to 6%).

The values for particle size reduction after wetting, cited above, are a compilation of values for sample nos. 1, and 7 in Table 1. Intermediate behavior is expected for Sample no. 4 because of its intermediate pore size. Wetting directly in PBS or 70% ethanol results in a significant decrease in porosity, and wetting via the stepwise processes results in retention of porosity after wetting.

The mechanism of porosity retention for the new wetting procedures deserves some attention. Without wishing to be bound by theory, surface tension of the wetting agent likely plays a role. It may be difficult to wet a dry collagen particle with a high surface tension liquid such as water or PBS. Instead of filling the pores in the interior of the sponge, which are initially filled with air and or water vapor, the liquid crushes the sponge. The force required for the liquid to penetrate the pores exceeds the compressive strength of the dry sponge. The surface tension of water is 75 dynes/cm², and that for ethanol is 22 dynes/cm². Ethanol can penetrate the pore without collapsing the structure. Once the pores are filled with liquid, the structure is not crushed by addition of a higher surface tension liquid to the liquid particle slurry.

Although one would expect that wetting directly in a high alcohol, water/alcohol mixture would also preserve porosity effects, a 70% alcohol/30% water solution, which has a surface tension of 26.3 dynes/cm2 (just slightly higher than for ethanol alone) resulted in a significant decrease in porosity upon direct addition. Therefore, the compressive strength of the dry sponge may be less than 26.3 dynes/cm².

Furthermore, pore size for the dry sponges should play a role. Reduction of porosity upon wetting should be inversely proportional to pore size, based on the explanation with respect to surface tension described above. This is apparent from measurement of particle size reduction upon wetting. The sponge with the smallest dry pores, 5μ to 100μ, exhibits the largest reduction, 67%, of porosity upon wetting directly into PBS. The sponge with the largest pores, μ200μ, exhibits a reduction of 32% upon wetting. Both values are measurements of the reduction in maximum particle diameter upon wetting. A 57% reduction of in cross-sectional area is also reported for the large pore sample, but the change in cross-sectional area may be expected to be more dramatic than that for maximum particle diameter. Moreover, results based on the confocal images are consistent with particle measurements.

In addition, collagen concentration is expected to have an effect on porosity reduction upon wetting.

In another embodiment, variations of the step wetting procedure are contemplated. The 1^{st} step involves wetting dry sponges with a low surface tension, water soluble liquid. Transformation to an aqueous medium may be accomplished in a continuous process or semi-continuous process, instead of a batch process. Aqueous mixtures may be caused to flow through sponges wetted with the non-aqueous, water soluble solvent.

Applications of Sponges of the Invention

The sponges of the present invention may be used for any application that could make use of the support structures of the invention, e.g., collagen support structures that substantially retain their structure upon wetting with an aqueous medium. In certain embodiments, the sponges of the present invention may be used in tissue composites, as resorbable carriers of biological materials including pharmaceuticals, or in chromatography devices.

A) Tissue Composites Prepared from Collagen Sponges of the Invention

In one embodiment, the invention is directed to improved tissue composites, e.g., biocompatible composites, prepared from the sponges of the invention, which overcome or minimize the problems associated with existing tissue repair systems and can be easily prepared and maintained in a sufficient quantity, and suitable shapes, to enable convenient treatment of tissues requiring repair. Further discussion of the methods of preparation of these tissue composites is contained in PCT Application Number PCT/US03/10439, which is hereby incorporated herein by reference.

As used herein, the term “composite” includes a substantially solid material that is composed of two or more discrete materials each of which retains its identity, e.g., physical characteristics, while contributing desirable properties to the composite. For example, in certain embodiments of the invention, the composite is produced by two biopolymers each having independent physical characteristics, e.g., degree of cross-linking or porosity. Composites of the invention typically include a biocompatible scaffold or sponge of the present invention and a biocompatible gel.

As used herein, the term “gel” includes materials that exist in a two-phase colloidal system consisting of a solid and a liquid in more solid form than liquid form, i.e., a semi-solid, of low porosity capable of retaining or immobilizing cells, while allowing the cells to proliferate. Accordingly, the gel is preferably formulated to allow diffusion of nutrients and waste products to, and away from cells, which promotes tissue growth following contact of a subject with a composite. In addition, the gel is preferably formulated to provide structural support to components of the composite, e.g., cells, during formation of the composite. The term gel is intended to include materials that function as a “glue” to retain components of the composite in their desired location during formation of the composite as well as maintain the structural integrity of the composite following preparation and initial implantation in a subject. This aspect is particularly advantageous for composites in which the scaffold comprises particulates.

Preferred materials for use in composites of the invention are biopolymers. As used herein, the term “biopolymer” includes biocompatible materials composed of one or more polymeric materials that are typically formed in a biological system or synthetically prepared from biologically available monomers. A biopolymer of the invention can be in the form of a solid, semi-solid, or liquid, and can be isolated from a biological system or synthetically prepared. Additionally, biopolymeric solidification of a solution can occur, e.g., by aggregation, coagulation, coacervation, precipitation, ionic interactions, hydrophobic interactions, or cross-linking. In one embodiment of the invention, the biopolymer is a cross-linked biopolymer. Cross-linking of the materials of the composite may be induced chemically, thermally (e.g., dehydrothermal cross-linking), or by radiation, e.g., ultraviolet. Cross-linking agents for chemical cross-linking include but are not limited to glutaraldehyde, formaldehyde and like aldehydes; hexamethylene diisocyanate, tolylene diisocyanate, and like diisocyanates; ethyleneglycol diglycidylether, and like epoxides; and carbodiimide hydrochlorides. In a specific embodiment of the invention, the biopolymer is thermally cross-linked (e.g., dehydrothermal cross-linking).

For use in vivo, particularly in human subjects, it is preferred that the materials that compose the composite include materials that are biocompatible with the subject. The term “biocompatible” includes materials that are compatible with a subject and are not toxic or deleterious to the subject. In certain embodiments of the invention, the biocompatible material is biodegradable, such that it degrades or decomposes following contact with a subject, e.g., human.

In specific embodiment, the biocompatible material of the composite is a biopolymer, e.g., as described above. Alternative biopolymers for use in the composites of the invention include complex coacervates. The term “complex coacervate” includes an aggregate, e.g., of colloidal droplets, held together by electrostatic attractive forces. Additionally, the aggregate may be hydrated, i.e., comprising water. In certain embodiments of the invention, the complex coacervate comprises calcium alginate and gelatin A, or calcium alginate. In one embodiment of the invention, a complex coacervate gel is prepared by contacting a biocompatible porous scaffold comprising a first component of the complex coacervate, e.g., a soluble form of calcium, with a biopolymer solution comprising a second component, e.g., sodium alginate, of the complex coacervate. (This is formulation described in example 16) The combination of the two components upon the combination of the scaffold with the biopolymer solution acts to solidify the biopolymer solution through coacervation and aggregation.

Additionally, other biopolymers for use in the composite include agarose and mixtures of agarose and gelatin A. Preferably, the melting point for a gel comprising agarose and gelatin A is lower than for a gel comprising agarose alone. In a specific embodiment, the agarose mixture is a low temperature melting agarose.

The term “alginate” includes the salt or ester of an insoluble colloidal acid (C₆H₈O₆)ₙ, which in the form of its salts is a constituent of the cell walls of brown algae. In certain embodiments, the alginate exists as a calcium salt, and is thus termed a calcium alginate. Alginate is a polysaccharide; which can be derived from brown seaweeds, composed of D-mannuronic and L-glucuronic acid monosaccharide subunits. While the sodium salt of alginate forms viscous solutions, alginate can form hydrated gels in the presence of divalent cations such as calcium due to cross-linking through the negatively charged carboxyl groups residing on the L-glucuronic acid residues. The viscosity of the uncross-linked solutions and thereby the mechanical strength of cross-linked gels can be influenced by altering the average chain length of the alginate or by altering the proportion of D-mannuronic acid and L-glucuronic acid residues within the polysaccharide. These factors may also influence the rate of resorption of the alginate. Alginate is commercially available, for example, from Kelco International Ltd. Waterfield, Tadworth, Surrey, UK.

The term “gelatin” includes a variety of substances (such as agar) resembling gelatin, e.g., glutinous material obtained from animal tissues by boiling, e.g., colloidal protein used as a food, in the art of photography, and in the art of medicine. Gelatin A is prepared by briefly treating pigskins with dilute acid followed by extraction with water at 50-100° C. The resulting gelatin A has a high isoelectric point (pI), and thus is positively charged at physiological pH.

The term “agarose” includes a polysaccharide obtained from agar, e.g., known in the art as a common supporting medium in gel electrophoresis. Agarose is commercially available, for example, from Sigma, Poole, England.

The term “gelling,”is well known in the art, and includes the act of becoming solid or thickened by chemical or physical alteration, thereby changing into a gel.

In another embodiment, the invention is directed to a multi-cellular composite comprising at least one first multi-functional unit (MFU), and at least one second MFU. In this embodiment, the multi-cellular composite contains at least one MFU that comprises a first biocompatible porous scaffold in contact with a first biocompatible gel seeded with a first population of cells wherein the gel is in contact with at least one surface of the scaffold.

The language “multi-cellular composite” includes composites of two or more cell populations. In preferred embodiments of the invention, at least one of the two or more cell populations is seeded in gel in desired compartments in the composite such that the cell types are located to provide a specific tissue function in a subject. For example, in one embodiment of the invention, the first population of cells comprises fibroblasts and the second population of cells comprises keratinocytes.

Other embodiments of the invention involve the preparation of tissue composites of different shapes or forms using composites of the invention. The composite can be shaped to corresponded to the desired tissue to be formed, e.g., soft tissue, e.g. skin, bone, an organ, e.g., cartilaginous tissue, e.g., a meniscus for a knee, an ear, a nose, or other tissue. The shape of the composite may be equally affected by the shape of the individual components of the composite, i.e., the scaffold or the gel. Molding the composite to the desired shape can be achieved by selecting the shape of either the scaffold or the gel. In one embodiment, the shape of the composite is a product of a mold in which either the scaffold or the gel or both the scaffold and the gel are formed. For example, after mixing the desired cell types, the gelling agent and the collagen scaffold at a condition that will retard the gelling of the mixture, the mixture can be injected or cast into a mold of the desired structure under appropriate conditions to facilitate gelling of the mixture to the desired structure.

In another embodiment of the invention, a composite is prepared on the surface of a mesh to facilitate transfer to a subject. Preferred mesh comprises a polymer that is not bioabsorbable, preferably having a pore size ranging from 3 to 216 microns in diameter. In one embodiment, a nylon mesh is be used to reduce shrinkage of the composite, particularly with composites containing fibroblasts. It has been determined that shrinkage of the composite during in vitro culture is analogous to wound contraction in vivo, and therefore, the mesh and the desired size of the collagen particulates in the composite may be used advantageously in reducing wound contraction, if any, in vivo. Additionally, the mesh may be used to assist in handling of the composite prior to implantation in a subject or to assist in forming the composite into a desired shape.

A composite or sponge of the invention may be affixed to the patient through grafting techniques known in the art, for example, such as described by J. Hansbrough et al. (Journal of Med. Assoc., vol. 262, No. 15, Oct. 20, 1989 pp. 2125-2130. J. Hansbrough, S. Boyce, M. Cooper, T. Foreman Burn Wound Closure With cultured Autologous Keratinocytes and Fibroblasts Attached to a Collagen-Glycosaminoglycan Substrate). Additionally, the composite may be affixed to the subject through gelatinization, or lamination, as described by Morota et al. in U.S. Pat. No. 6,051,425.

For use in tissue repair, composites of the invention include one or more cell populations. Typically, the composite is seeded with cells of at least one cell type. The language “seeded with cells” includes a distribution of cells retained or immobilized within a material that contributes to the composite, e.g., the gel or scaffold. In certain embodiments, the distribution of cells is retained or immobilized in, for example, the gel, the scaffold, or both. The distribution of cells may be of a single type or of multiple types, e.g., as in the multi-cellular composites. In certain embodiments of the invention, the distribution of cells is a uniform distribution. In an embodiment where both the scaffold and the gel are seeded with cells, the cells may be selected for a specialized function in vivo (e.g., dermal and epidermal cells for skin repair) or be seeded with cells for independent function. Cells are selected and added to the material such that the composite can perform its intended function. Cells for use in the composites can be primary cells harvested from a donor, cultured cells, e.g. allowed to proliferate in vitro, or cryopreserved cells. A cellular composites may also be produced using the appropriate methods of the invention.

The language “cells contained in,” for example, in the expression, “the cells contained in the scaffold,” refers to a dispersion of cells in a biocompatible material, e.g., biopolymer, or adsorption of the cells and/or cell solution onto the surfaces of a biocompatible material. In contrast, the language “seeded with cells,” refers to retention, or immobilization, and placement of cells within a biological material.

Cell types for use in the methods and compositions of invention include, for example, fibroblasts, keratinocytes, and stem cells. Cells for use in the methods and compositions of invention include primary cells, cultured cells and cryopreserved cells.

Examples of cell types for use in the methods and compositions of invention include but are not limited to epidermal and dermal cells (e.g., keratinocytes or fibroblasts), muscle cells (e.g., myocytes), cartilage cells (e.g. chondrocytes), bone forming cells (e.g., osteoblasts), epithelial cells (e.g., corneal cells, tracheal cells, or mucosal cells), endothelial cells, pleural cells, ear canal cells, tympanic membrane cells, peritoneal cells, gingiva cells, neural cells, hepatocytes, pancreatic cells, cardiac cells, and stem cells.

Cells for use in the methods and compositions of invention can be isolated from a tissue biopsy or bone marrow sample from a subject, using methods known to those skilled in the art. If insufficient cell numbers are available at isolation, the cells can be allowed to proliferate in culture prior to seeding into a composite of the invention. During cell growth and proliferation, the cells can be cultured as a monolayer on a tissue culture treated substrate and maintained in tissue culture medium such as Dulbeccos Modified Eagle's Medium supplemented with, for example, between 1 and 20% fetal calf serum or autologous human serum. Alternatively, the cells can be cultured in serum free medium supplemented with mitogens on tissue culture plastic modified by the immobilization of specific attachment factors. In another approach, isolated cells can be seeded at a specified seeding density within alginate beads and cultured in tissue culture medium supplemented with serum or mitogenic growth factors. The cells can be isolated by dissolving the beads in a sodium citrate saline solution followed by collagenase digestion. The cells can be cultured within a suitable bioreactor.

In a particular embodiment for skin repair, cells are obtained from skin sample from a subject to be treated (autologous) or from donor tissue (allogenic). Skin samples are treated with trypsin to separate the epidermis from the dermis (Eisinger, M. Method in Skin Research, Editor D. Skerrow, (1985) pp 193). The epidermis is minced and treated with trypsin to release keratinocytes. The keratinocytes are then cultured until confluence using standard methods. In certain embodiments, the keratinocyte cells are cultured as single cell suspensions until confluence. Alternatively, in a preferred embodiment, the keratinocyte cells are seeded as single cell suspensions and cultured until confluence.

Primary cultures of fibroblast cells for use in accordance with the present invention may be prepared using standard methods such as, for example, the method disclosed in “A specific collagenase from Rabbit fibroblasts in monolayer culture,” Journal of Biochemistry (1974) 137, 373-385. Preferably, primary cultures of fibroblasts are prepared as follows. A dermal sample is cut up into 1 mm cubes and is suspended in a solution of collagenase buffered with Tris-HCl pH 7.4. A suitable collagenase is Clostridium histolyticum collagenase. The dermal sample is preferably suspended in solution at a concentration of 1 microgram/mL. The suspension is incubated and then centrifuged at 1,500 rev/sec to remove the cells from solution. The suspension is preferably incubated for 30 minutes. The cell pellet is washed with DMEM and the number of fibroblasts is determined with a haemocytometer. The viability of the fibroblast is determined by dye exclusion using Trypan Blue. The above culturing method also surprisingly yields other dermal epithelial cells that have a potential to develop into sweat glands or other skin cell types. An additional source of fibroblasts and keratinocytes includes neonatal foreskin, in which the cells can be isolated by standard protocols as described above.

Additionally, the present invention contemplates a continuous process for preparing sheet-like single layer and multiple layer engineered tissue matrices comprising cells, a particulate biopolymer scaffold, and a biopolymer gel and the composites made thereby. In one embodiment, the process may further comprise the following steps:
(a) mixing an aqueous dispersion of a particulate biopolymer scaffold, e.g., comprising collagen, with cells dispersed in a solution of a gellable biopolymer, e.g., a collagen solution, at a temperature at which the gellable biopolymer solution will not gel;
(b) casting the mixture of cells, particulate biopolymer scaffold, and biopolymer gel onto a film, e.g., a polymer film, in a continuous web process; and
(c) heating the mixture to a temperature at which the gellable biopolymer solution gels.
   Prior to the mixing step, the process may further comprise one or more of the following steps: (1) culturing cells on a particulate biopolymer scaffold in an aqueous medium that supports cell growth to produce an aqueous dispersion of cells attached to the particulate biopolymer scaffolds; (2) preparing a dispersion of a particulate biopolymer scaffold and cells in a solution of a gellable biopolymer at a temperature at which the gellable biopolymer solution will not gel. In certain embodiments, the film is porous and excess aqueous medium is removed from the mixture of cells, biopolymer scaffold, and gellable biopolymer solution prior to gellation of the gellable biopolymer solution.

An additional embodiment of the invention is directed to a process for producing multiple layer matrices comprising
preparing a first layer prepared by the continuous process described above for preparing sheet-like single layer; and
casting a second layer onto the first layer, wherein the second layer is prepared by the continuous process described above for preparing sheet-like single layer; comprises cells dispersed in a biopolymer gel; or comprises an aqueous dispersion of cells,
   wherein the second layer is cast onto the first layer in a continuous web process. An example of the preparation of such composites is described in Example 17B. As such, composites prepared by this process are within the contemplation of the present invention with or without the porous film. Furthermore, the dry collagen sponges of these composites may be further wetted by the processes described herein.
   B) Delivery Devices Prepared From Collagen Sponges of the Invention

In one embodiment, the invention is directed to an enclosure comprising the wetted spherical and/or non-spherical particulates, of the present invention. The term “enclosure” as used herein, is defined as a mold or shaped container that is capable of receiving the sponges of the present invention. In certain embodiments, the invention is directed to an “enclosure device,” which is defined as an enclosure capable of containing a composition, such that the enclosure becomes at least an integrated component of the resulting composition, i.e., a composite prepared in a mold containing a mesh anchoring portion, or a wound sealed at the exposed surface with a film or fabric or some other suitable cover that encloses the wound and becomes integrated with the final composition.

In specific embodiments, the enclosure and/or enclosure device comprises a film or fabric, e.g., porous fabric, or some other suitable cover that contacts the composition, e.g., the sponges of the present invention. The enclosed composition may be an engineered tissue composition and or a carrier device, e.g., a drug delivery device. In certain embodiments, at least one face of the enclosure device is living tissue.

The shape of the enclosure or mold may be tailored for the end use. For example, the shape could be an element/characteristic of the tissue to be replaced/regenerated. Compositions comprising the wetted spherical and/or non-spherical particulates are cast into the mold. If an engineered tissue is to be constructed, the mold and contents may be cultured in a nutrient medium, e.g., in vitro or in vivo.

Another embodiment of an enclosure is a “mold” containing wetted spherical and/or non-spherical particulates, cells, and a “vascular system,” i.e., a plumbing system that provides nutrients, e.g., a system of blood vessels is a vascular system that supplies a flow of nutrients. The vascular system may be designed to mimic that in a human or animal. A further embodiment of this invention is the use of particulates seeded with cells. The seeded particulates are cultured in a bioreactor to produce seeded particulates with a high cell density. These are placed in the enclosure comprising the vascular system and cultured in vitro or in vivo. Advantageously, this embodiment overcomes the problems associated with the delivery of nutrients to thick sections of engineered tissue.

In another embodiment, the invention is an enclosure comprises a carrier device comprising the wetted spherical and/or non-spherical particulates, of the present invention and an additional component. In certain embodiments, the additional component is a microorganism, e.g., bacteria, cells, e.g., a drug, pharmaceutical agents, e.g., small and large molecules, cells modified to express a desired pharmaceutical agent antibiotic, growth factor, steroid, spermicidal agent, and the like, as well as combinations thereof. Accordingly, the carrier devices may be comprised of solely the sponges and the additional agents or may be comprised of sponges as part of a composite (which can also be referred to as micro-carrier composites). The carrier devices of the invention may be cellular, e.g., a cell-based drug delivery device, or acelluar.

In certain embodiments, each particle of the carrier is encased in a complex coacervate gel. It should be noted that the process of preparing such complex coacervates, as described herein, may be used to coat medical devices, e.g., stents, which are to be implanted into a subject, and such an application is within the scope of this invention.

The additional component of the carrier device may be incorporated into the collagen particle before or after cross-linking, e.g., addition of the additional component may occur at the dispersion stage or after dehydrothermally cross-linking.

Another embodiment of the invention is an aqueous dispersion or slurry comprising the spherical and/or non-spherical particulates, of the present invention, and a microorganism.

Yet another embodiment of the invention is a medical sealant comprising an aqueous dispersion or slurry comprising the spherical and/or non-spherical particulates, of the present invention.

C) Chromatography Devices Prepared from Collagen Sponges of the Invention

In another embodiment, the invention is a chromatography media comprising the wetted spherical and/or non-spherical particulates of the present invention. Chromatography devices of the invention may be monolithic in nature or may be composed of packed particles, which are useful for chromatographic separations, e.g., size exclusion or affinity. In certain embodiments, the sponges of the present invention may also be useful as a filter media.

Another embodiment of the invention is a device comprising a container enclosing a monolithic interpenetrating network comprising a continuous polymer network and a continuous network of voids. The container may be any receptacle capable of holding the sponges of the invention, e.g., both particulate and non-particulate (e.g., sheets, e.g., producing the monolithic chromatographic medium), which would be acceptable for use in the chromatographic arts, e.g., glass or steel. The polymer may be a naturally occurring biopolymer, e.g., a protein, polysaccharide, or lipid, which may also be cross-linked, e.g., dehydrothermally cross-linked, chemically cross-linked, or cross-linked by radiation. In specific embodiments of the chromatographic device, the biopolymer is collagen. The polymer may be water-swellable.

In one embodiment, the invention is directed to a method of producing a device comprising a container enclosing a monolithic interpenetrating network comprising a continuous polymer network and a continuous network of voids comprising the following steps:
producing an aqueous solution and/or a dispersion of a polymer;
filling a tube with the solution and/or dispersion of a polymer;
freezing the solution and/or dispersion of a polymer in the container; and
lyophilizing the container filled with the frozen solution and or dispersion of a polymer.

In certain embodiments, the aqueous solution or dispersion further comprises an organic solvent. In additional embodiments, the aqueous solution or dispersion is frozen in a bath, e.g., liquid nitrogen, maintained at a temperature below the freezing point of the solution and or dispersion of the polymer.

Another embodiment of the invention is a method of producing a device comprising a container enclosing a monolithic interpenetrating network comprising a continuous polymer network and a continuous network of voids comprising the following steps:
producing an aqueous solution and/or a dispersion of a polymer;
freezing the solution and or dispersion of a polymer in the shape of the container;
lyophilizing the shaped, frozen solution and/or dispersion of a polymer to form a monolithic interpenetrating network comprising a continuous polymer network and a continuous network of voids into a container.
inserting the shaped, lyophilized, monolithic interpenetrating network into the container
sealing the monolith into the container to insure that the monolith is in contact with interior wall of the container.

Additionally, the contact between the monolith and the container wall may be established by hydrating the lyophilized monolith inside the tube.
In particular embodiments, the lyophilized monolith is further subjected to the steps of:
wetting in a non-aqueous water soluble solvent and then
exposing the wetted cross-linked scaffold to a gradient of solvent mixtures comprising the non-aqueous solvent and water, starting with a high concentration of the non-aqueous solvent and ending with water.
   Definitions

In addition, several additional terms have been used in and throughout the specification; for convenience the definitions of these terms are shown below:

The language “biological material” includes a material or agent that is biocompatible with a subject, e.g., an animal, e.g., a human. Examples of biological materials include, but are not limited to water, buffered solutions, saline, nutrient solutions supportive of cell growth, cells, cell cultures, proteins, amino acids, cytokines, e.g., lymphokines, blood products, hormones, antibodies, e.g., monoclonal, toxins, toxoids, vaccines, e.g., viral, bacterial, endogenous and adventitious viruses, and pharmaceutical agents, e.g., pharmaceutical drugs. In one embodiment of the invention, the biological material is a biological solution.

The term “casting” is well known in the art, and includes the process by which a material is formed into a shape to by pouring liquid into a mold and letting harden without pressure. Another method of casting involves the formation of a spherical shape by pumping a liquid through a small orifice and casting spherical droplets in air. In one embodiment of the invention, the hardening of the material is performed through temperature changes. In another embodiment of the invention hardening of the material is performed via complex coacervation. In certain embodiments of the invention, the casting of the scaffold is accomplished by exposure to low temperatures, e.g., liquid nitrogen. It should be noted that the concept of casting is distinct from the concept of hardening, wherein the latter is incorporated into the process of casting.

The language “contact” or “contacting” includes the union or junction of surfaces. The union may be made through a single point, in a region, i.e., surface, or in separate points or separate regions. The term “surface” as used herein includes the outer periphery, exterior, or upper boundary of a material. In certain embodiments, the term surface is used herein to describe a sheet structure, e.g., a scaffold in the form of a sheet, which is generally planar, e.g., a planar, or curved, two-dimensional locus of points (as in the boundary of a three-dimensional region). In certain embodiments, contact of one surface is made with a primary face, e.g., a first primary face, of another surface. The language “primary face” includes surfaces of sheet structures that are comparatively larger than other surfaces of the sheet structure. Several examples of materials in contact are shown in FIGS. 1-5 of PCT Application PCT/US03/10439.

The term “continuous web process” is one in which a liquid or liquid-like material is coated onto a web (film, paper, foil, or fabric) in a continuous process. In one embodiment, a liquid is the ungelled mixture of cells, gellable solution, and particulate sponges, and the web is a porous nylon fabric; gellation occurs after coating as a result of a change in temperature.

The language “improving a condition of a tissue” includes growth of new tissue, protection of the tissue, e.g., from injury, e.g., infection, prevention of fluid loss, and tissue support to improve conditions for natural repair mechanisms of the subject. In one embodiment, contacting the tissue of a subject with a composite of the invention returns the tissue to a healthy state.

The language “multi-functional unit (MFU)” is intended to include distinct geographical and functional units (e.g., a unit with a distinct biological activity/function, e.g., a unit distinctly positioned for the growth of separate populations of cells) of a multi-cellular composite, wherein each functional unit may comprise a gel, a scaffold, a biological material, e.g., a cell population, or any combination thereof. For example, in certain embodiments of the invention, scaffold and gel combine to form one distinct multi-functional unit of a multi-cellular composite. In certain other embodiments, scaffold, gel, and cells are combined to form a single multi-functional unit. It should be understood that the inclusion of a biological material in a single MFU is not limited to a single biological material, e.g., a single MFU may contain more than one type of cell in a cell population.

The language “nutrient solution supportive of cell growth” includes solutions that contain nutrients, e.g., amino acids or growth factors supportive of cell growth. Optionally, the nutrient solution can contain cells.

The term “particulate,” “microsphere,” and “particulate sponge” are used interchangeably, as defined herein, and includes materials, e.g., biopolymers, which are particle in nature, e.g., relatively minute, small, or discrete. In the present invention, the term “particulate” is intended to include both spherical and non-spherical particulates.

The term “sheet” is intended to cover sponges of shapes that are not encompassed within the term particulate, i.e., non-particulate sponges.

The term population” includes a group of individual objects, or items from which samples are taken for statistical measurement.

The term “porous” includes materials having pores through which substances can pass. In certain embodiments of the invention, the scaffold component of the composite has an average pore size that allows for cell growth, for example, a porosity that allows nutrients and waste products to diffuse through the material. In another embodiment, the sponge has an average pore size that allows for the in-growth of cells.

The language “subject” includes animals e.g., mammals, e.g., dogs, cats, horses, pigs, cows, sheep, goats, rodents, mice, rats, rabbits, squirrels, bears, and primates e.g., chimpanzees, gorillas, and humans, as well as transgenic non-human animals. Preferably, the subject is a human, e.g., a human requiring treatment of a tissue, e.g., wound repair.

The language “surface porosity” refers to the size (area and diameter) of the pores on the surface of the sponge, i.e., the pores that immediately accessible to the a biological material that would be added to the sponge, e.g., an aqueous solution.

The term “tissue” includes cellular material capable of forming a collective entity. In one embodiment, a tissue is a collection or aggregation of morphologically and functionally similar cells. The term “wound” includes bodily injuries, including those which result in injury to a tissue, e.g., skin, e.g., a dermal wound.

The terms “treating” and “treating a tissue or wound”are intended to include improving at least one condition of a tissue or wound, and tissue augmentation, i.e., plastic surgery, e.g., lip injections of composites.

The language “volume fraction” of component, is defined as:

$$\frac{Volume of component}{Total Volume of composition}$$

Accordingly, the volume fraction of a component is a number between 0 and 1.

The term “washing” is related to the term wetting, and includes the process of wetting a material with a liquid that has already been already bee made wet, e.g., to replace a non-aqueous water soluble solvent with an aqueous medium. The term “wetting,” is well known in the art, and includes the act of making a material wet. For example, in one embodiment of the invention involves the wetting of a biocompatible porous scaffold with a biological material, e.g., a biological solution. In addition, the wetting (or washing) may be performed in a batch or continuous process.

Exemplification

General Overview of Exemplification

Insoluble type I bovine collagen from SIGMA was used for most formulations. One formulation may be prepared with collagen from a human source supplied by Sigma. Another formulation may be prepared with recombinantly produced collagen from Fibrogen. Collagen, acetic acid, and water were mixed at 6000 rpm for 30 min at a temperature <25 C with a lab scale Silverson rotor/stator mixer. The mixture was stored overnight in a cooler above the freezing point of the dispersion. See Examples below for specific formulations.

To prepare dry spherical collagen sponges, a collagen dispersion was metered with a peristaltic pump through a vibrating no. 22 needle dropwise into a bath of liquid nitrogen. Frozen specimens were lyophilized for 5 days at a pressure <60×10⁻³ MBAR.

The lyophilized sponges were dehydrothermally cross-linked at 120 C at <1 torr for 3 days. In one embodiment, spherical sponges were prepared by casting droplets into a pentane bath at −15 C.

In addition, in the methods for the preparations of the sponges, collagen concentration was varied from 1 mg/ml to 10 mg/ml, and acid concentration was varied from 0.5% to 0 5% by weight. See Table 1 for formulations and results. Formulations comprising high collagen and or low acid concentrations could not be pumped through the needle due to high viscosity and/or large particle size. At low collagen concentration and low acid concentration significant deformation occurred upon lyophilization and the particles were not spherical.

In certain embodiments, optimum collagen and acid concentrations for dry sphere production, in liquid nitrogen or pentane, are 5 mg/ml and 5% by weight, respectively. The sponges exhibited a highly porous open cell structure. Spheres cast in liquid nitrogen, No. 1, exhibit a maximum pore size of 5 u to 10 u. Spheres cast in pentane at −15 C, no. 4, have a maximum pore size of 20 u to 30 u. Both were used in a wetting experiment described below.

To prepare dry non-spherical particulates, a collagen dispersion was poured into ice cube trays. The trays containing the dispersion were placed in a foam polystyrene; container with a lid. The whole assembly was placed in a freezer set to −20 C. The assembly was slowly cooled to generate a large pore size. The dispersion was chilled for at least 2 days, at which point the dispersion is frozen.

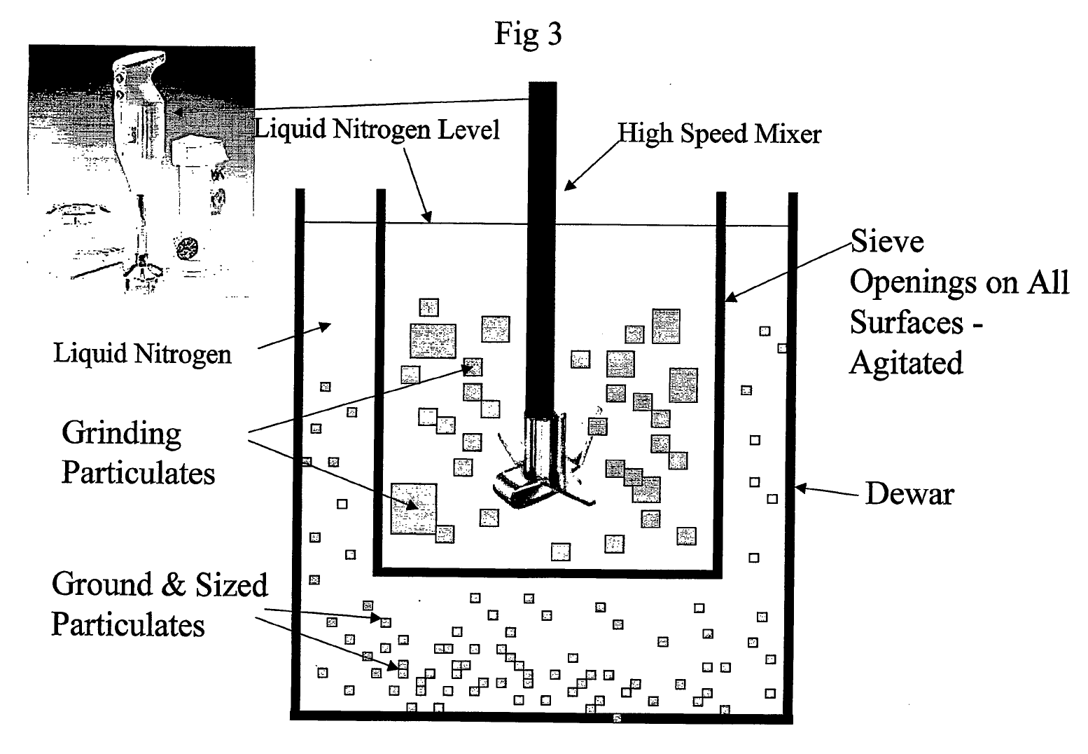

Frozen cubes were quickly removed from the cooler, split in half, and added to a stainless steel sieve suspended in a liquid nitrogen bath. The sieve was agitated with a shaker. The cubes, immersed in liquid nitrogen, were ground with a high speed kitchen type mixer, such that ground particles smaller than the sieve fall through. See FIG. 3 for schematic of apparatus. The ground frozen particles may be separated into additional fractions with additional sieves. Frozen particle fractions were lyophilized for 5 days at a pressure <60×10⁻³ MBAR. The lyophilized sponges are dehydrothermally cross-linked at 120 C at <1 torr for 3 days. Collagen concentration was varied between 5 mg/ml and 50 mg/ml and acid concentration was varied from 0.5% to 5% by weight. See Table 1 for formulations and results.

Formulations comprising 50 mg/ml collagen were extremely viscous, and frozen, lyophilized and dehydrothermally cross-linked materials prepared from these dispersions were either non-porous or exhibit closed cell structures. Open cell sponges were produced with a dispersion comprising 5 mg/ml collagen and 5% acid. The particles with a maximum dry pore size of 200μ, e.g., sample no. 7, are used for the wetting experiments described below.

Other variations of non-spherical particles may also be prepared. In one embodiment, the non-spherical particles were made by casting large droplets of collagen dispersion into liquid nitrogen. In another embodiment, the non-spherical particles were made by casting and freezing in ice cube trays at −80 C. In addition, preparation conditions for the non-spherical particles may be the same as described above for spherical particles.

Dry sponges were imaged with SEM. Representative SEM images of dry particles are depicted in FIG. 2 for sample nos. 1, 4, and 7 in Table 1. Maximum pore size was estimated visually from SEM photomicrographs. Average pore size was also measured with Image Pro Plus 4.5 and the dry pore size measurements are reported in Table 1. The procedure for average pore size measurements is described in a further example below.

Dry sponges may be wetted by various procedures. One embodiment involves wetting with a series of nine ethanol-phosphate buffer (PBS) mixtures shown below as 1 through 9. The collagen sponges were first added to a flask containing absolute ethanol. Reduced pressure was applied for a short duration to facilitate wetting. The flask was sealed and shaken until the particles sank. Most of the ethanol was decanted off and the second alcohol/PBS mixture was added. The flask was shaken again until the particles sink. This procedure was repeated for the remaining alcohol/PBS mixtures.

1. 100% ethanol

2. 85% ethanol/15% PBS

3. 75% ethanol/25% PBS

4. 65% ethanol/35% PBS

5. 60% ethanol/40% PBS

6. 50% ethanol/50% PBS

7. 30% ethanol/70% PBS

8. 15% ethanol/85% PBS

9. 5% ethanol/95% PBS

For a 2 step process, collagen sponges were first added to a flask containing absolute ethanol. Reduced pressure was applied for a short duration to facilitate wetting. The flask was sealed and shaken until the particles sank. Most of the ethanol was decanted off and PBS was added. The flask was shaken again until the particles sank.

Collagen sponges may also be wetted directly into either PBS or 70% ethanol in water. Reduced pressure was applied for a short duration to facilitate wetting. The flask was sealed and shaken until the particles sank.

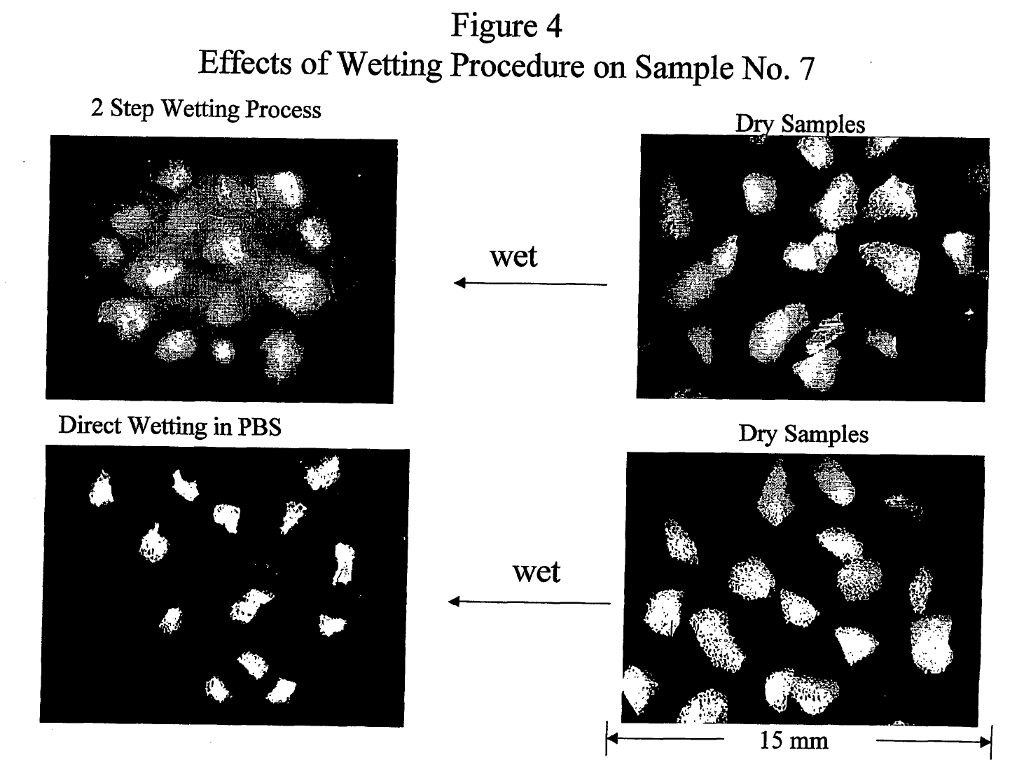

Measurement of particle size before and after wetting is an indirect method of retention of porosity upon wetting. FIG. 4 presents a comparison of particle size reduction for two different wetting procedures for sample no. 7. The particles wetted via a 2 step procedure shrink very little. In contrast, the particles wetted directly in the PBS aqueous solution shrink significantly.

Two types of measurements were made for various particles before and after wetting by different methods (See Table 1). Maximum particle diameters were measured before and after wetting for a population of 10 to 20 particles with a stereo microscope fitted with a graded eyepiece for sample 1. Maximum particle diameter was measured before and after wetting with, Image-Pro Plus, an image analysis software package, for sample 7A. Cross-sectional area was measured before and after wetting with Image Pro Plus for samples 7B and 7C. The order of porosity reduction upon wetting, as measured via change in particle diameter or particle cross-sectional area is as follows: direct in PBS (23% to 670%)>direct in 70% ethanol (410%)>>2 step (−0.2% to 17.7%) ≧9 step process (−1.6% to 6%). In conclusion, the results indicate that direct wetting in PBS or 70% alcohol results in significant particle shrinkage, while wetting via a multi-step processes results in little shrinkage.

Porosity after wetting was also evaluated with confocal laser scanning microscopy. Sponges were stained with Alexa Fluor 488 carboxylic acid dye solution 1 mg/ml PBS (Molecular Probe Cat # A-20000). A Zeiss LSM400 microscope was used and the emission at 488 was observed. Images of the 3 different particles wetted via the nine step process and directly into PBS are shown in FIG. 1. The porosity is significantly reduced for wetting directly in PBS versus the multistep procedure for samples 1 and 4. For example, note the comparison of samples 1 (a) to (d), and samples 2 (b) to (e). Examination of sample 7 demonstrates a significant, but less pronounced reduction in pore size for wetting directly in PBS versus the multistep procedure. For example, samples (c) to (f) may be compared. The less dramatic effect for sample 7 versus samples 1 and 4 may be attributed to the larger pore size for sample no. 7.

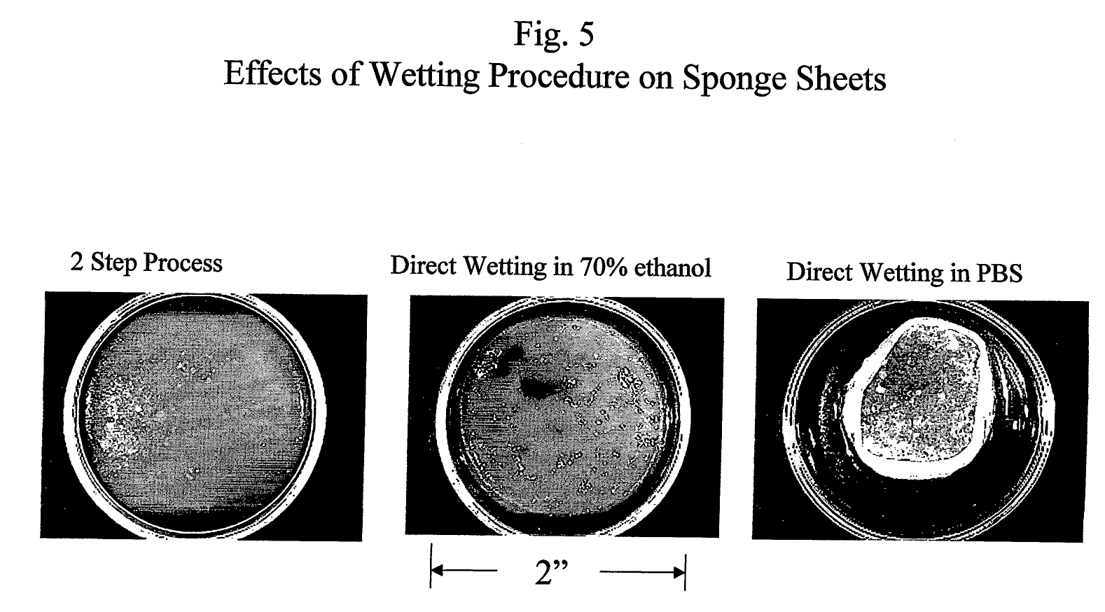

Images of a sheet sponge, frozen at −20 C, wetted via the two step process, directly wetted in 70% alcohol, and directly wetted in PBS are shown in FIG. 5. Note the significant reduction in the size of the disk for direct wetting in PBS. There is moderate reduction in the size of the disk for direct wetting in 70% alcohol. There is virtually no shrinkage for the stepwise process.

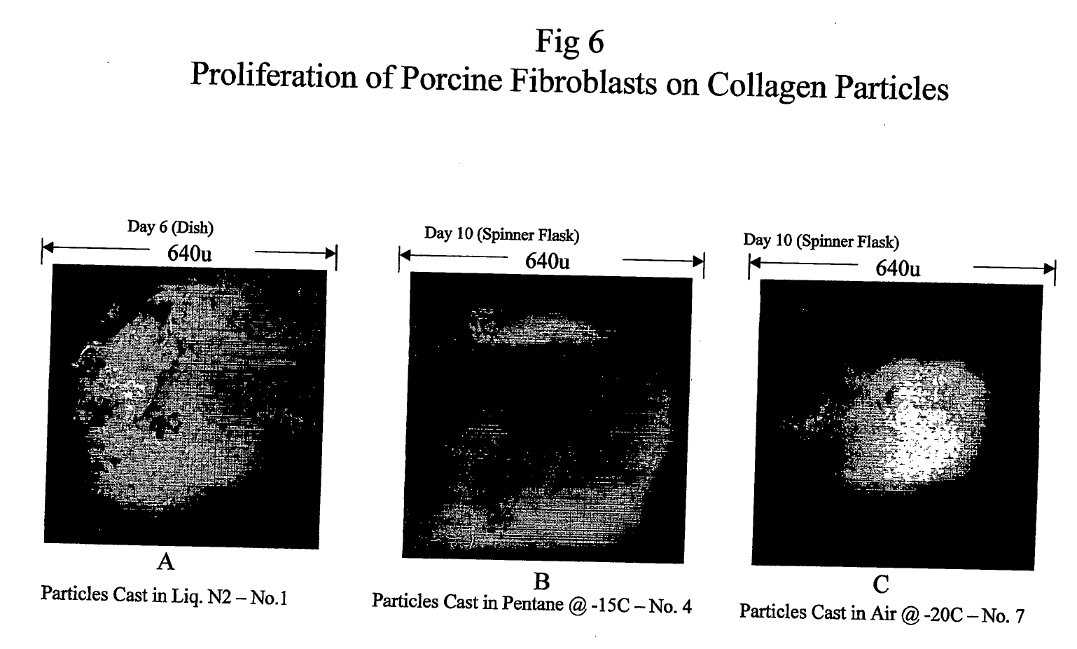

To ascertain the ability of these particles to support cell growth in vitro, an equivalent volume of each type of collagen particles, labeled sample nos. 1, 4, and 7 in FIG. 6, were used. Porcine fibroblasts, (3×10⁶) were mixed with the washed particles in a 6-well plate insert with a 0.4 micron mesh at the bottom in a 100-mm petri dish. The cells and particles in the insert were incubated in 2 ml of F12/DMEM medium containing 15% fetal calf serum, supplements and antibiotics at 37 C in a CO₂ incubator for two hours. The whole insert was subsequently covered with culture medium and further incubated at 37 C for the duration indicated. Alternatively, the collagen particles with the cells were transferred to a spinner flask after overnight incubation at 37 C in a 100-mm dish.

The proliferation of the fibroblasts was determined by confocal microscopy. As indicated in FIG. 6, all three types of particles support the proliferation of the cells. In particular, higher cell density is observed from cultures (FIGS. 6B and 6C) incubated for 10 days in spinner flasks when compared to that (FIG. 6A) incubated for 6 days in a 100 mm Petri dish. Accordingly, the degree of proliferation depends on the duration of the incubation as well as the type of culture vessels used for the study.

TABLE 1

| | | | | | | | | | Max Particle | | | |
|---|---|---|---|---|---|---|---|---|---|---|---|---|
| | | | | | | | | | Reduction after Wetting | | | |
| | | | | | Glacial | | Dry Max | Dry Avg | | | Direct | Direct |
| | | | Casting | Collagen | Acetic Acid | Pore | Pore Size | Pore Size | 9 | 2 | in | in 70% |
| | Dry Particulate | Casting | Temperature | Conc. | Conc. | Morphology | Visual | Image Pro | Step | Step | PBS | ethanol |
| No. | Shape | Medium | (F.) | (mg/ml) | (% vol.) | Dry Particles | (u) | (u) | (%) | (%) | (%) | (%) |
| 1 | spherical | Liquid N2 | | 5 | 5 | open | 5 to 10 | 2.33 | −1.6³ | 0.6³ | 67³ | 41³ |
| 2 | deformed sphere² | | | 3 | 5 | open | | | | | | |
| 3 | deformed sphere² | | | 1 | 5 | open | | | | | | |
| 4 | spherical | Pentane | −15 | 5 | 5 | open | 20 to 30 | 3.41 | | | | |
| 5 | deformed sphere² | | −15 | 3 | 5 | open | | | | | | |
| 6 | deformed sphere² | | −15 | 1 | 5 | open | | | | | | |
| 7A | Non-Spherical | Air | −20 | 5 | 5 | open | 200 | 15.4 | | 7.6² | 32.3² | |
| 7A | Non-Spherical | Air | −20 | 5 | 5 | open | 200 | | | −0.2¹ | 57.2¹ | |
| 7B | Non-Spherical | Air | −20 | 5 | 5 | open | 200 | | | 13² | 23.2² | |
| 7B | Non-Spherical | Air | −20 | 5 | 5 | open | 200 | | | 17.7¹ | 49¹ | |
| 7C | Non-Spherical | Air | −20 | 5 | 5 | open | 200 | | 6.0¹ | | 48¹ | |
| 8 | Non-Spherical | Air | −20 | 5 | 0.5 | less open | | | | | | |
| 9 | Non-Spherical | Air | −20 | 50 | 5 | non-porous | | | | | | |
| 10 | Non-Spherical | Air | −20 | 50 | 0.5 | closed | | | | | | |
| 11 | Disk Shaped | | | | | | 200 | | | | | |
| | Sheet | | | | | | | | | | | |
| 12 | Non-Spherical | Liquid N2 | | 5 | 5 | open | 10 to 20 | 3.95 | | | | |
| 13 | Non-Spherical | Air | −80 | 5 | 5 | open | 5 to 10 | | | | | |

¹Reduction in cross-sectional area after wetting as measured with Image Pro Plus
²Reduction in particle maximum diameter as measured with Image Pro Plus
³Reduction in particle diameter as measured with a light microscope

The invention is further illustrated by the following examples, which should not be construed as further limiting.

A. Spherical Particulate Sponges

### EXAMPLE 1

### Effects of Formulation and Process Variables on Dry Sponge Porosity and Shape

Example 1 demonstrates the effects of collagen concentration, acetic acid concentration, collagen solubility, freezing temperature, and freezing medium on porosity and particle shape of the particulate sponges of the invention.

### EXAMPLE 1B

### Effects of Freezing Conditions and Collagen Concentration on Morphology of Dry Spherical Particles

Materials and Methods

The effects of collagen concentration and cooling conditions on the pore size of dry, spherical particles were evaluated. Formulations listed in the table below were produced and imaged. For nos. 1 to 3 collagen spheres were prepared as follows. Insoluble, type I, bovine collagen from SIGMA was used for all samples. Collagen, acetic acid, and water were mixed at 6000 rpm for 30 min at a temperature <25 C with a lab scale Silverson rotor/stator mixer. The mixture is stored overnight in a cooler above the freezing point of the dispersion. The resulting dispersion was added dropwise through a vibrating no. 22 needle into a bath of liquid nitrogen. Frozen specimens were lyophilized for 5 days at a pressure <60×10⁻³ MBAR. The lyophilized sponges were dehydrothermally cross-linked at 120 C at <1 torr for 3 days.

For samples 4 to 6, spheres are prepared as described above with the exception that the dispersion is added dropwise to a stirred pentane bath maintained at −15 C to affect freezing.

For sample 7, specimens are prepared as described above with the exception that droplets of the dispersion are placed onto a silicone coated plastic film. The droplets spread out to form disk shaped structures. The whole assembly was placed, at room temperature, into a polystyrene foam insulated container. The container was then placed into a −20 C freezer to affect slow cooling. Frozen specimens were lyophilized for 5 days at a pressure <60×10⁻³

MBAR. The lyophilized sponges were dehydrothermally
cross-linked at 120 C. at <1 torr for 3 days.

| | Collagen | Acetic | Deionized | | Coolant | | |
|---|---|---|---|---|---|---|---|
| | Concentration | Acid | Water | | Temp | Largest | Particle |
| No | (mg/ml) | (ml) | (ml) | Coolant | (C.) | Pore Size u | Shape |
| 1 | 5 | 10 | 190 | Liquid N2 | | 5 | spherical |
| 2 | 3 | 10 | 190 | Liquid N2 | | 5 to 10 | sl. spherical |
| 3 | 1 | 10 | 190 | Liquid N2 | | 5 to 10 | collapsed |
| 4 | 5 | 10 | 190 | Pentane | −15 | 20 to 30 | spherical |
| 5 | 3 | 10 | 190 | Pentane | −15 | 20 to 30 | sl. spherical |
| 6 | 1 | 10 | 190 | Pentane | −15 | collapsed | collapsed |
| 7 | 5 | 10 | 190 | air | −20 | >50 | |
| 23 | 5 | 10 | 190 | Pentane | −70 | 5 to 10 | |

The specimens were imaged via SEM, wherein the largest pore size was visually estimated from the photomicrographs.
Results and Conclusions:
Results are summarized as follows:

Cooling conditions have the biggest impact on pore size. The largest pores (>50μ) were obtained for samples cooled at the slowest rate, slowly in air at −20 C. Intermediate pore size was obtained at the intermediate cooling rate, −15 C in pentane. The smallest pores were obtained for the fastest cooling rate, liquid nitrogen. Small pores were also obtained for samples cast in pentane at −70 C. These pores were similar in size to those for spheres cast in liquid nitrogen.

For samples made in liquid nitrogen, pore size was slightly affected by collagen concentration with the 3 mg/ml and 1 mg/ml having slightly larger pore size than that for the 5 mg/ml.

For samples made in liquid pentane, pore size was similar at collagen concentrations of 5 and 3 mg/ml. Pores were collapsed at a collagen concentration of 1 mg/ml.

Collagen concentration has the largest impact on particle shape. This comparison is only made for samples 1 to 6 since these are spherical after freezing. Sample 7 is disk shaped after freezing. Structures best approximating a sphere were obtained at a collagen concentration of 5 mg/ml. Misshapen structures were obtained at a collagen concentration of 1 mg/ml. At intermediate collagen concentration the particles are somewhat spherical.

Qualitatively, frozen collagen spheres comprising the lowest collagen concentration shrank and disfigured the most in the lyophilization process than spheres comprising the higher collagen concentration. All samples were approximately spherical after freezing and prior to lyophilization. Additional shrinkage was not apparent during dehydrothermal cross-linking.

### EXAMPLE 1C

### Effects of Freezing Conditions on Pore Size Distribution of Dry Spherical Sponges—Image Analysis Technique

Materials and Methods:

Pore size measurements were made for digital SEM photomicrographs obtained for samples 1 and 4 from example 1B using the computer software program Inage Pro Plus 4.5 (available from Media Cybernetics). The protocol for the measurement process using Image Pro Plus 4.5 is as follows:

From the main menu

(a) open image;

(b) select measure, select calibration, select spatial, set the spatial calibration and close the calibration window;

(c) select measure again from the main menu, select count/size, select measurement, select select measurements, from the drop down box select diameter (max.) and area as the measurements to be made, and select “OK”;

(d) from the count/size window make sure that automatic dark objects, measure objects, and apply filter ranges are all checked;

(e) select the count button; and

(f) to see data select view and then measurements from the count/size window.

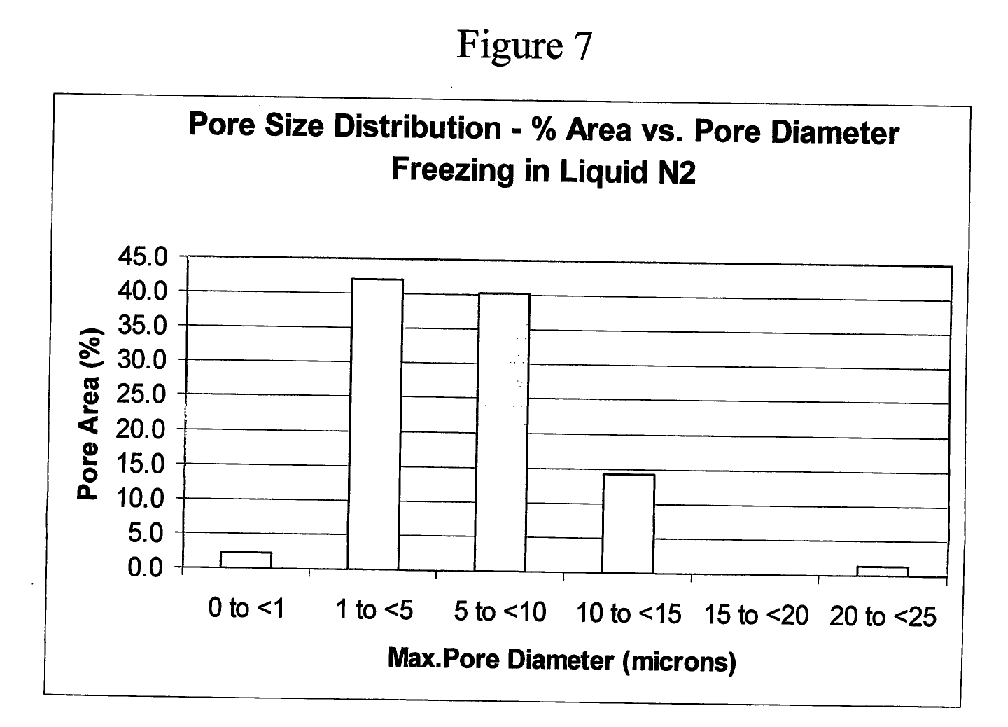

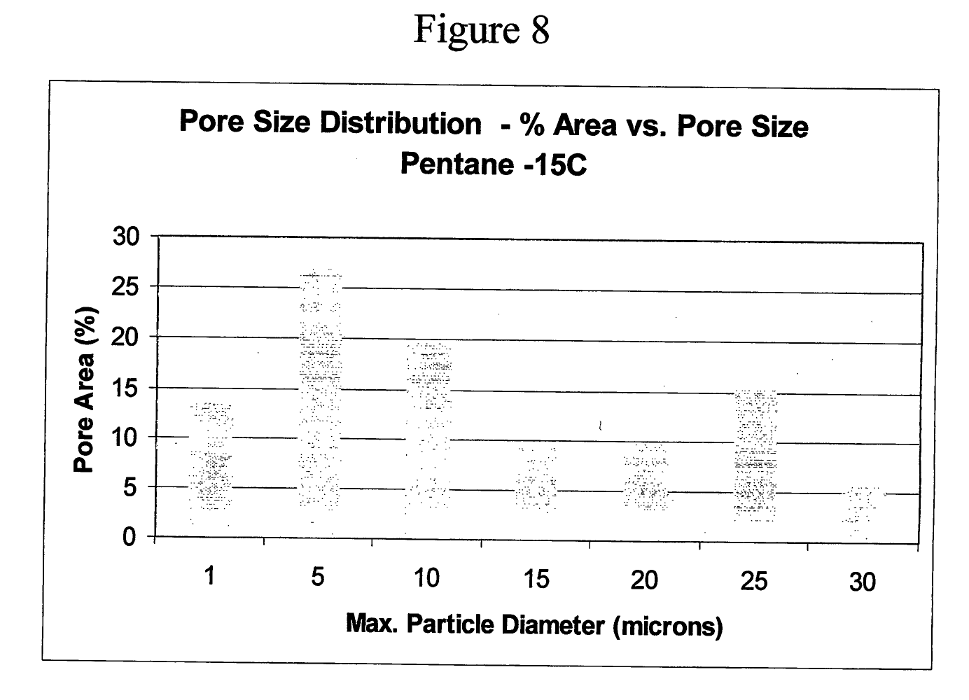

The area and maximum particle diameter data were used to construct a plot of pore area % as a function of pore diameter for samples 1 and 4 shown in FIGS. 7 and 8. The average maximum particle diameter was also calculated. Results are shown below.

Results and Conclusions:

Results are summarized as follows

For particles frozen in liquid nitrogen, about 15% of the total area of the surface pores is occupied by pores ≧10 microns in diameter. In contrast, for particles frozen in pentane at −15 C, about 50% of the total area of the surface pores is occupied by pores ≧10 microns in diameter. As such, it is evident from the results described herein that the particles frozen in pentane at −15 C can be differentiated from those particles cast in liquid nitrogen by pore size distribution, i.e., particularly by using the method presented herein.

### EXAMPLE 1 E

### Effects of Acid and Collagen Concentration and Freezing Conditions on Maximum Pore Diameter and Pore Area of Dry Spherical Sponges—Image Analysis Technique

Materials and Methods:

The effects of freezing conditions, acid and collagen concentration on sponge pore size were evaluated to determine parameters which contribute to the largest pore size for spherical particulates. Collagen dispersions were prepared with insoluble bovine collagen. Collagen, acetic acid, and water were mixed at 6000 rpm for 30 min at a temperature <25 C. with a lab scale Silverson rotor/stator mixer. The mixture is stored overnight in a cooler above the freezing point of the dispersion. The resulting dispersion was added dropwise through a vibrating no. 22 needle into a bath of pentane at −15 C or liquid nitrogen. Frozen specimens were lyophilized for 5 days at a pressure <60 MBAR. The lyophilized sponges were dehydrothermally cross-linked at 120 C at <1 torr for 3 days. Spheres that could be prepared were imaged via SEM. Magnification is in the range of 1000× to 2000×. This magnification range should be used for analyzing particles with mean max. pore diameter in the range of 2 u to 4 u (or max pore size of ˜20 u).

For a dispersion comprising 5 mg/ml collagen and 5% glacial acetic acid two lots of spheres were cast in liquid nitrogen and 5 lots were cast in pentane at −15 C. One lot each were cast in pentane at −15 C for the following combinations

5 mg/ml collagen/5% glacial acetic acid

5 mg/ml collagen/3.5% glacial acetic acid

3 mg/ml collagen/2.5% glacial acetic acid

5 mg/ml collagen/2.5% glacial acetic acid

−5 mg/ml collagen/0.5% glacial acetic acid

Multiple photos were imaged for each lot. Image Pro Plus 4.5 was used to analyze the digital SEM photomicrographs. The protocol for the measurement process using Image Pro Plus 4.5 is as follows:

From the main menu

(a) open image;

(b) select measure, select calibration, select spatial, set the spatial calibration and close the calibration window;

(c) select measure again from the main menu, select count/size, select measurement, select select measurements, from the drop down box select diameter (max.) and area as the measurements to be made, and select “OK”;

(d) from the count/size window make sure that automatic dark objects, measure objects, and apply filter ranges are all checked;

(e) select the count button; and

(f) to see data select view from the count/size box and then select statistics to see the average values for max. diameter and area.

Results of pore size measurements are shown below. Average
values for max. particle diameter and average area are shown.

| | | | | | | | Mean |
|---|---|---|---|---|---|---|---|
| | | | | | | Mean | Max. |
| | | Temp | collagen | acid | | Area | Diam. |
| No | Coolant | C. | mg/ml | wt % | Mag | Microns² | microns |
| 28-2 | pentane | −15 | 5 | 3.5 | 1000 | 7.4 | 3.4 |
| 28-3 | pentane | −15 | 5 | 3.5 | 2000 | 7.1 | 3.2 |
| 28-5 | pentane | −15 | 5 | 3.5 | 1000 | 7.2 | 3.3 |
| 28-6 | pentane | −15 | 5 | 3.5 | 2000 | 3.7 | 2.4 |
| Avg. | | | | | | 6.35 | 3.075 |
| 15-2 | Liq. N2 | | 5 | 5 | 1700 | 2.22 | 1.97 |
| 15-4 | Liq. N2 | | 5 | 5 | 1700 | 2.76 | 2 |
| 4B | Liq. N2 | | 5 | 5 | 1300 | 3.92 | 2.66 |
| 4IIA | Liq. N2 | | 5 | 5 | 1300 | 5.3 | 2.7 |
| Avg. | | | | | | 3.55 | 2.33 |
| 1B | pentane | −15 | 5 | 5 | 1300 | 14.8 | 4.18 |
| 1-IIB | pentane | −15 | 5 | 5 | 1300 | 15.7 | 3.83 |
| 1-111B | pentane | −15 | 5 | 5 | 1300 | 17.6 | 3.56 |
| 24-2 | pentane | −15 | 5 | 5 | 1300 | 8.4 | 3.7 |
| 24-3 | pentane | −15 | 5 | 5 | 1300 | 4.7 | 2.8 |
| 24-4 | pentane | −15 | 5 | 5 | 1300 | 7.3 | 3.7 |
| 24-6 | pentane | −15 | 5 | 5 | 1300 | 14.7 | 4.4 |
| 24-7 | pentane | −15 | 5 | 5 | 1300 | 6.2 | 3.1 |
| 24-9 | pentane | −15 | 5 | 5 | 1300 | 8.5 | 3.6 |
| 26-2 | pentane | −15 | 5 | 5 | 1000 | 5.10 | 2.97 |
| 26-4 | pentane | −15 | 5 | 5 | 2000 | 3.36 | 2.2 |
| 26-5 | pentane | −15 | 5 | 5 | 1000 | 12.6 | 2.6 |
| 30-2 | pentane | −15 | 5 | 5 | 1000 | 8.8 | 3.8 |
| 30-4 | pentane | −15 | 5 | 5 | 1000 | 8.1 | 3.5 |
| 37-2 | pentane | −15 | 5 | 5 | 1000 | 13.5 | 2.8 |
| 37-5 | pentane | −15 | 5 | 5 | 1000 | 8.1 | 3.8 |
| Avg. | | | | | | 9.84 | 3.41 |
| 29-2 | pentane | −15 | 3 | 2.5 | 1000 | 4.9 | 3 |
| 29-3 | pentane | −15 | 3 | 2.5 | 1000 | 4.9 | 2.9 |
| 29-5 | pentane | −15 | 3 | 2.5 | 1000 | 4 | 2.8 |
| 29-6 | pentane | −15 | 3 | 2.5 | 1000 | 3.5 | 2.5 |
| Avg. | | | | | | | 2.8 |

| | | | | | |
|---|---|---|---|---|---|
| 2 | pentane | −15 | 5 | 2.5 | can not be cast |
| 3 | pentane | −15 | 5 | 0.5 | can not be cast |

Results and Conclusions

Spheres cast in pentane for formulation comprising 5 mg/ml collagen and 5% acid, exhibit the largest values for mean maximum pore diameter and mean pore area. The pore size of these spheres is significantly larger than that for the spheres cast in liquid nitrogen. For example, the avg. max. diameters (i.e., average of averages) are 3.4μ and 2.3μ for the pentane and liquid nitrogen samples, respectively. As such, it is evident from the results described herein that the particles frozen in pentane at −15 C can be differentiated from those particles cast in liquid nitrogen by pore size distribution, i.e., particularly by using the method presented herein.

In contrast, casting the spheres with lower acid or collagen in pentane at −15 C does not result in larger pores.

Formulations 2 and 3 listed at the end of the table shown above could not be cast into spheres. Collagen particle size in the dispersion is too big to pump through no. 22 needle

### EXAMPLE 1F

### Spheres from Human Collagen

Collagen from human placenta Type VI from Sigma is used. A mixture of 5 mg/ml in 5% acetic acid are mixed at 6000 rpm for 30 min at a temperature <25 C with a lab scale Silverson rotor/stator mixer. The mixture is stored overnight in a cooler above the freezing point of the dispersion. The resulting mixture is added dropwise through a vibrating no. 22 needle into a bath of liquid nitrogen. Frozen specimens are lyophilized for 5 days at a pressure <60×10⁻³ MBAR. The lyophilized sponges are dehydrothermally cross-linked at 120 C at <1 torr for 3 days. The spheres are wetted by the stepwise wetting procedures described in other examples

### EXAMPLE 1G

### Spheres from Recombinantly Produced Collagen

Recombinant Human Collagen I, 3 mg/ml in 10 mM HCl, from Fibrogen is added dropwise through a vibrating no. 22 needle into a bath of liquid nitrogen. Frozen specimens are lyophilized for 5 days at a pressure <60×10⁻³ MBAR. The lyophilized sponges are dehydrothermally cross-linked at 120 C at <1 torr for 3 days. The spheres are wetted by the stepwise wetting procedures described above

### EXAMPLE 1H

### Collagen Sponges Comprising Drug Added to Dispersion

A mixture of a water soluble or a water insoluble drug and 5 mg/ml of insoluble type I bovine collagen from SIGMA in 5% acetic acid is prepared. The mixture is mixed at 6000 rpm for 30 min at a temperature <25 C with a lab scale Silverson rotor/stator mixer. The mixture is metered with a peristaltic pump through a vibrating no. 22 needle dropwise into a bath of liquid nitrogen. Frozen specimens are lyophilized for 5 days at a pressure <60×10-3 MBAR. The lyophilized sponges are dehydrothermally cross-linked at 120 C at <1 torr for 3 days. The spheres are wetted by the stepwise wetting procedures described in previous examples.

### EXAMPLE 1I

### Collagen Sponges Wetted with Solution of Water Soluble Drug

Wetted sponges obtained in Example 14 are wetted with water, and transferred to a 0.2μ filter unit. The water is removed via filtration to a point where the wetted particulates are packed, but without a visible layer of liquid on top of the packed sponges. A solution of a water soluble drug is carefully added so that solution rests on top of layer of sponges. Drainage is allowed to occur until the liquid level just reaches the top of the layer of spheres.

### EXAMPLE 1J

### Collagen Sponges Comprising Drug Added to Dispersion—Chemically Attached

A mixture of water soluble or water insoluble drug, a chemical agent to chemically bond the drug to collagen, and 5 mg/ml insoluble type I bovine collagen from SIGMA in 5% acetic acid is prepared. The mixture is mixed at 6000 rpm for 30 min at a temperature <25 C with a lab scale Silverson rotor/stator mixer. The mixture is metered with a peristaltic pump through a vibrating no. 22 needle dropwise into a bath of liquid nitrogen. Frozen specimens, are lyophilized for 5 days at a pressure <60×10-3 MBAR. The lyophilized sponges are dehydrothermally cross-linked at 120 C at <1 torr for 3 days. The spheres are wetted by the stepwise wetting procedures described in previous examples.

### EXAMPLE 3

### Measurement of Changes in Spherical Sponge Diameter after Wetting—Light Microscope Technique

Materials and Methods

9 Step Gradient

Dry dehydrothermally cross-linked spheres were produced as for no. 1 in the overview of exemplification. These were used for all of the measurements in example 3. The spheres were wetted by the same 9 step gradient wetting procedure as for described in the overview of exemplification. Nine spheres were randomly selected for this experiment. The maximum diameter of the dry spheres was measured and the diameter of the spheres was measured after the gradient wetting. These measurements were made manually with a stereo microscope. See table below. Note that average diameters are nearly identical.

| | Dry Diameter | Diameter after |
|---|---|---|
| | dry | Gradient Washing |
| | 1.9 | 1.9 |
| | 1.8 | 2.3 |
| | 1.8 | 1.7 |
| | 1.8 | 1.8 |
| | 1.9 | 1.8 |
| | 1.7 | 1.6 |
| | 1.4 | 1.9 |
| | 2.1 | 1.9 |
| | 2 | 1.7 |
| Average | 1.81 | 1.84 |

2 Step Gradient

The diameters of 13 dry spheres were measured. The same spheres were wetted in 99.8% ethanol with the application of reduced pressure for a few minutes to facilitate wetting. Excess ethanol was removed from the spheres wetted with ethanol and the spheres were wetted in phosphate buffer solution. Incubate in phosphate buffer solution until all spheres sink to the bottom of the container. Diameters were measured. Note that average diameters are nearly identical.

| | Dry diameter | Wet Diameter |
|---|---|---|
| | (mm) | (mm) |
| | 1 | 1.2 |
| | 2.5 | 1.2 |
| | 1.5 | 2.5 |
| | 1.5 | 2.5 |
| | 1.2 | 2 |
| | 2.5 | 2.5 |
| | 2.5 | 2 |
| | 1.2 | 1.5 |
| | 2.5 | 1.5 |
| | 1.5 | 1.2 |
| | 1.5 | 1 |
| | 1.2 | 1.2 |
| | 1 | 1.2 |
| Average | 1.66 | 1.65 |

Direct Wetting in Medium

The maximum diameters of 16 dry spheres were measured and the results are shown below. The same 16 spheres were wetted directly in PBS. Reduced pressure was applied to facilitate wetting. Note the significant reduction in diameter after wetting directly in PBS.

| Dry Spheres | Spheres in PBS |
|---|---|
| Diam. mm | Diam. mm |
| 1.2 | 0.5 |
| 1.2 | 0.4 |
| 1.2 | 0.4 |
| 1.2 | 0.6 |
| 2.2 | 0.5 |
| 2.6 | 0.5 |
| 2 | 0.4 |
| 1 | 0.5 |
| 1 | 0.6 |
| 1 | 0.5 |
| 2 | 0.4 |
| 2 | 0.5 |
| 1 | 0.5 |
| 1 | 0.4 |
| 1.4 | 0.5 |
| 1.4 | 0.5 |
| Ave. 1.46 | Ave. 0.48 |
| | ˜67% shrink |

Direct Wetting in 70% Ethanol

The maximum diameters of 14 dry spheres were measured and the results are shown below. The same 14 spheres were wetted directly in 70% ethanol/30% PBS. Reduced pressure was applied to facilitate wetting. Note the significant reduction in diameter after wetting directly in 70% ethanol/30% PBS.

| Dry Spheres | Spheres in 70% Ethanol |
|---|---|
| Diam. mm | Diam. mm |
| 2 | 1 |
| 2.5 | 1 |
| 2 | 0.8 |
| 1.8 | 1.2 |
| 1.2 | 1 |
| 1 | 2 |
| 2 | 1 |
| 2 | 0.8 |
| 1.2 | 0.6 |
| 2 | 0.8 |
| 1.4 | 1 |
| 1.4 | 0.6 |
| 1.2 | 0.8 |
| 1.4 | 0.8 |
| Ave. 1.65 | Ave. 0.95 |
| | ˜41% shrink |

Results and Conclusions:
Results are summarized as follows

The 9 step gradient wetting process results in substantially no shrinkage. Furthermore, particles wetted with the two-step gradient, comprising wetting in ethanol and then in medium, also resulted in substantially no shrinkage.

In contrast, wetting directly in medium or directly in 70% ethanol/30% PBS results in considerable shrinkage.

### EXAMPLE 4A

### Definition of Spherical and Measurements

The term “spherical” is defined as follows: 250% of particles in a population exhibit a roundness value of 1 to 1.2 using the equation
Roundness=(Perimeter²)/(4*pi*area)
Dry Spheres

Dry spheres described as sample no. 4 in example 1B were used for the measurements. Roundness was measured using a digital image of a population of spheres and Image Pro Plus 4.5. The protocol is outlined in example 5A1. All roundness values for this dry sphere population were between 1 and 1.2

Wet Spheres

Dry spheres described as sample no. 4 in example 1B were used. These were subjected to the 9 step wetting procedure described in examples 2 and 3 and the roundness was measured using a digital image of a population of spheres with Image Pro Plus 4.5 as described for dry spheres. As some spheres were in contact with one another, the image was adjusted prior to making measurements using the split object tool in Image Pro Plus 4.5. Sixty % of spheres exhibit roundness values in the range of 1 to 1.2

Conclusions:

The spherical particle shape is maintained when a stepwise wetting procedure is used. In contrast, the particles directly wetted in an aqueous medium are highly misshapen and roundness increases dramatically as compared with particles exposed to a stepwise gradient, e.g., the nine-step gradient described herein.

B. Non—Spherical Particulate Sponges

### EXAMPLE 5

### Production of Dry Non Spherical Particulate Collagen Sponges—Freezing in Air

Materials and Methods:

A formulation for a collagen dispersion is shown below

For 200 ml of dispersion:

| Collagen Concentration | 5 | mg/ml |
|---|---|---|
| Acetic Acid Concentration | 5% | |
| Weight of Collagen | 1 | gr |
| Volume of Glacial acetic acid | 10 | ml |
| Volume of DDW | 190 | ml |
| Final volume of preparation | 200 | ml |

The formulation was mixed for 30 min at 6000 rpm with a lab scale Silverson rotor stator mixer. The mixture was stored overnight in a cooler above the freezing point of the dispersion. The dispersion was poured into ice cube trays with dimensions of 141×1″×1.75″. Each tray was filled about ⅔ to ¾ of the available volume. The trays containing the dispersion were placed in a foam polystyrene container with a lid. The whole assembly was placed in a freezer set to −20 C. The assembly was slow cooled to generate a large pore size. The dispersion was chilled for at least 2 days, at which point the dispersion is frozen.

Three frozen cubes were quickly removed from the cooler, split in half with a stainless steel knife, and added to a stainless steel Dewar containing ˜6 oz. of liquid nitrogen. The cubes in a liquid nitrogen medium were ground with a hand held high speed kitchen mixer. Grinding was done in 2-30 sec. periods.

The resulting dispersion of frozen particles in liquid nitrogen was poured into a series of sieves that were immersed in liquid nitrogen. The array was agitated to affect separation of particles according to size. Alternatively, the grinding and separation may be done in a single step, in liquid nitrogen, as shown in FIG. 3. Frozen particle fractions were lyophilized for 5 days at a pressure <60×10⁻³ MBAR. The lyophilized sponges were dehydrothermally cross-linked at 120 C at <1 torr for 3 days.

The dehydrothermally cross-linked particles were wetted in multistep processes to preserve the porosity as described in the overview of the exemplification.

### EXAMPLE 5A-1

### Measurement of Particle Max. Diameter, Particle Cross-Sectional Area, and Particle Roundness—Image Analysis Technique

A visible light microscope was used to produce a photomicrograph of wet or dry particle, which was used for these measurements. The protocol for the measurement process using Image Pro Plus 4.5 is as follows:

From the main menu

(a) open image;

(b) select measure, select calibration, select spatial, set the spatial calibration and close the calibration window;

(c) select measure again from the main menu, select count/size, select measure, select select measurements, from the drop down box select diameter (max.), area, and roundness as the measurements to be made, and select “OK”;

(d) from the count/size window make sure that automatic-bright objects, measure objects, and apply filter ranges are all checked;

(d alternative) from the count/size window make sure that manual, measure objects, and apply filter ranges are all checked; then select colors, activate pen, use pen to fill in bright objects, and close

(e) select the count button; and

(f) to see data select view and then select data and or statistics; average values are shown in statistics.

To evaluate the percentage change in cross-sectional area for a dry versus a wet particulate, the average cross-sectional area for a population of dry particles was measured. The same population of dry particles was wetted. The average cross-sectional area of the wetted particulates was then measured. The percentage change in cross-sectional area (ACA) was then calculated using the following formula:
ACA=100×(Avg dry cross-sectional area−Avg. wet cross-sectional area)/Avg dry cross-sectional area

To evaluate the percentage change in average maximum diameter for a dry versus a wet particulate, the average maximum diameter for a population of dry particles was measured. The same population of dry particles was wetted. The average maximum diameter of the wetted particulates was then measured. The percentage change in average maximum diameter (AMD) was then calculated using the following formula:
AMD=100×(avg. max. diameter dry−avg. max. diameter wet)/avg. max. diameter dry

### EXAMPLE 5A

### Characterization of Wet Non Spherical Particulate Collagen Sponges—Particle Size Distribution—Method A

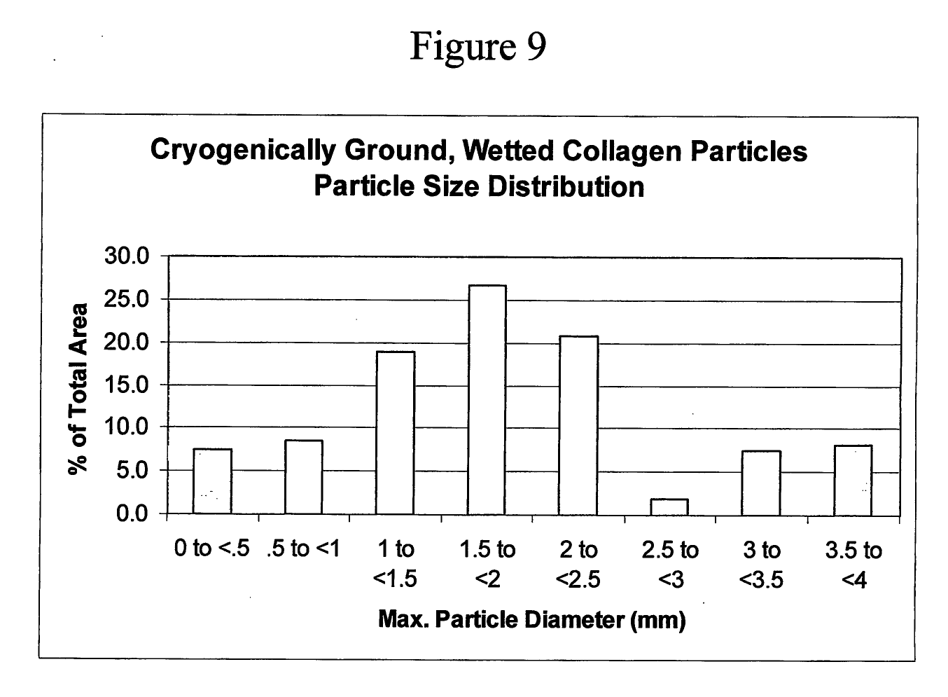

Grinding and separation were done simultaneously. The fraction passing through a 1.5 mm sieve and retained on a 0.5 mm sieve was collected, lyophilized, and cross-linked. The dry particles formed aggregates and appeared to be charged. The particles were wetted in a stepwise process as described above. The dispersion of the wetted particles was then agitated to break up aggregates of particles. An aqueous dispersion of the particles was imaged. The photomicrographs were analyzed using Image Pro Plus 4.5 for Max. particle diameter, particle area, and particle roundness as described in 5A-1 The resulting particle size distribution is shown in FIG. 9, in which the % of the total particle area was plotted in comparison to particle size, and demonstrated that greater than 50% of the population has a max. particle diameter between 1 mm to 2.5 mm. The average roundness was 4.7

Note that some particles are larger than the pore size of the sieve that was used (1.5 mm). One possible explanation is that the particles may bond together during the cross-linking process. A second possible explanation stems from the concept that it is difficult to completely break up aggregates of particles.

### EXAMPLE 5B

### Characterization of Dry Non Spherical Particulate Collagen Sponges—Avg. Max. Pore Diameter and Avg. Pore Area—Image Analysis Method

Image Pro Plus 4.5 was used to measure the average maximum pore diameter and average pore area for dry particles from photomicrographs. The protocol for the measurement process using Image Pro Plus 4.5 is as follows:

From the main menu

(a) open image;

(b) select measure, select calibration, select spatial, set the spatial calibration and close the calibration window;

(c) select measure again from the main menu, select count/size, select measurement, select select measurements, from the drop down box select diameter (max.) and area as the measurements to be made, and select “OK”;

(d) from the count/size window make sure that automatic dark objects, measure objects, and apply filter ranges are all checked;

(e) select the count button; and

(f) to see data select view and then measurements from the count/size window.

For sponges with an average max. diameter in the range of 10μ to 25μ (max pore diameter of 100μ) a magnification of 200× to 400× was used for the digital images. For sponges with a max pore diameter of about 10-20μ a magnification of 1000× was used for the pore size analysis. The results are shown in the table below. See Example 5 for preparation of the particles in table below

| | | | | | | | Mean |
|---|---|---|---|---|---|---|---|
| | | | | | | Mean | Max. |
| | | | | | | Area | Diam. |
| No | Coolant | Temp | Collagen | Acid | Mag | Microns² | Microns |
| 31-4 | air | −20 | 5 | 5 | 200 | 219.3 | 19.1 |
| 31-6 | air | −20 | 5 | 5 | 200 | 275 | 18.9 |
| 35-2 | air | −20 | 5 | 5 | 350 | 93.4 | 10.6 |
| 35-5 | air | −20 | 5 | 5 | 350 | 155 | 13.1 |
| Avg. | | | | | | 185.7 | 15.4 |

Additionally, samples prepared by the methods of Example 6 were measured and the results are shown in table below.

| | | | | | | | Mean | Mean |
|---|---|---|---|---|---|---|---|---|
| | | | Collagen | Sieve | | | Area | Max. Diam. |
| No | Coolant | Temp | Mg/ml | Size | acid % | Mag | Microns² | microns |
| 51-2 | Liq. N2 | | 5 | 2 to 3 | 5 | 1000 | 12.6 | 4.1 |
| 51-3 | Liq. N2 | | 5 | 2 to 3 | 5 | 1000 | 9.6 | 3.8 |
| Avg. | | | | | | | 11.1 | 3.95 |
| 52-2 | Liq. N2 | | 5 | .5 to 2 | 5 | 1000 | 9.8 | 3.7 |
| 52-3 | Liq. N2 | | 5 | .5 to 2 | 5 | 1000 | 10.3 | 3.8 |
| Avg. | | | | | | | 10.05 | 3.75 |

As shown in the tables above the mean area of the particulates ranged from 10 to 85 mm², and the mean maximum diameter ranged from about 3μ to 16μ in the particulates examined

### EXAMPLE 5C

### Preparation and Characterization of Wet Non Spherical Particulate Collagen Sponges—Particle Size—Method B

A formulation for a collagen dispersion is shown below

For 200 ml of dispersion:

| Collagen Concentration | 5 | mg/ml |
|---|---|---|
| Acetic Acid Concentration | 5% | |
| Weight of Collagen | 1 | gr |
| Volume of Glacial acetic acid | 10 | ml |
| Volume of DDW | 190 | ml |
| Final volume of preparation | 200 | ml |

The formulation is mixed for 30 min at 6000 rpm with a lab scale Silverson rotor stator mixer. The mixture is stored overnight in a cooler above the freezing point of the dispersion. The dispersion is poured into ice cube trays with dimensions of 1″×1″×1.75″. Each tray is filled about ⅔ to ¾ volume. The trays containing the dispersion are placed in a foam polystyrene container with a lid. The whole assembly is placed in a freezer at 20 C. The intent is to have slow cooling to generate a large pore size. The dispersion is chilled for, at least, 2 days at which point the dispersion is frozen.

Three frozen cubes are quickly removed from the cooler, split in half with a stainless steel knife, and added to a 5 mm stainless steel sieve suspended in a liquid nitrogen bath. The sieve is agitated with a shaker. The cubes, immersed in liquid, nitrogen are ground with a high speed kitchen type mixer. See FIG. 3. Ground particles, smaller than 5 mm fall through the sieve. The ground frozen particles are separated, while immersed in liquid nitrogen, into four fractions using a series of 3 additional sieves:

(a) 3 to 5 mm

(b) 2 to 3 mm

(c) 5 to 2 mm

(d) <0.5 mm

Frozen particle fractions were lyophilized for 5 days at a pressure of <60×10⁻³ MBAR. The lyophilized sponges were dehydrothermally cross-linked at 120 C at <1 torr for 3 days. The dry particles formed aggregates and appeared to be charged.

The particle fractions were wetted in a stepwise process as described above. The dispersion of the wetted particles was then agitated to breakup aggregates of particles. Digital images of the wetted particles were recorded. Four images were recorded for each of the 2 largest particle fractions. The photomicrographs were analyzed using Image Pro Plus 4.5 and the maximum average diameters were measured. The protocol for the measurement process using Image Pro Plus 4.5 is as follows:

From the main menu

(a) open image;

(b) select measure, select measurements;

(c) from Features box select creating line;

(d) manually, measure max. particle diameter for each particle in image using this tool; and

(e) to see data select feature tab.

Raw data and averages are shown below for the 2 largest fractions passing through the 3 to 5 and retained on the 2 to 3. (The 2^{nd} largest fraction is that passing through the 2 to 3 and retained on the 0.5 to 2.)

Maximum particle Diameter (mm)

| | | | |
|---|---|---|---|
| No. | 3 to 5 mm sieve | No. | 2 to 3 mm Sieve |

| | | | |
|---|---|---|---|
| 1 | 6.27 | 1 | 3.85 |
| 2 | 2.73 | 2 | 5.36 |
| 3 | 4.82 | 3 | 6.98 |
| 4 | 3.67 | 4 | 2.08 |
| 5 | 7.27 | 5 | 4.36 |
| 6 | 19.82 | 6 | 1.60 |
| 7 | 2.31 | 7 | 2.19 |
| 8 | 3.27 | 8 | 3.05 |
| 9 | 3.59 | 9 | 1.20 |
| 10 | 4.83 | 10 | 5.22 |
| 11 | 3.76 | 11 | 2.22 |
| 12 | 6.23 | 12 | 3.70 |
| 13 | 5.01 | 13 | 1.00 |
| 14 | 3.22 | 14 | 1.53 |
| 15 | 2.76 | 15 | 2.20 |
| 16 | 3.90 | 16 | 2.26 |
| 17 | 2.34 | 17 | 1.50 |
| 18 | 1.58 | 18 | 1.70 |
| 19 | 5.22 | 19 | 3.53 |
| 20 | 4.44 | 20 | 1.98 |
| 21 | 3.74 | 21 | 2.07 |
| 22 | 4.21 | 22 | 4.98 |
| 23 | 3.77 | 23 | 1.93 |
| 24 | 4.54 | 24 | 1.77 |
| 25 | 3.80 | 25 | 4.80 |
| 26 | 2.68 | 26 | 1.95 |
| 27 | 7.50 | 27 | 2.61 |
| 28 | 2.44 | 28 | 2.74 |
| 29 | 3.25 | 29 | 3.37 |
| 30 | 8.49 | 30 | 9.98 |
| 31 | 2.20 | 31 | 6.77 |
| 32 | 5.29 | 32 | 3.42 |
| Avg. | 4.53 | 33 | 2.97 |
| | | 34 | 0.77 |
| | | 35 | 1.76 |
| | | 36 | 2.12 |
| | | 37 | 13.49 |
| | | 38 | 5.71 |
| | | 39 | 2.12 |
| | | 40 | 1.92 |
| | | 41 | 2.16 |
| | | 42 | 4.40 |
| | | 43 | 1.91 |
| | | 44 | 2.24 |
| | | 45 | 1.56 |
| | | 46 | 2.07 |
| | | 47 | 2.26 |
| | | 48 | 1.69 |
| | | 49 | 3.64 |
| | | 50 | 1.40 |
| | | 51 | 2.22 |
| | | 52 | 4.76 |
| | | 53 | 2.25 |
| | | 54 | 3.88 |
| | | 55 | 7.70 |
| | | 56 | 2.80 |
| | | 57 | 2.02 |
| | | Avg. | 3.22 |

### EXAMPLE 6

### Production of Dry Non Spherical Particulate Collagen Sponges—Freezing in Liquid Nitrogen

Materials and Methods:

A formulation for a collagen dispersion is shown below

For 200 ml of dispersion:

| Collagen Concentration | 5 | mg/ml |
|---|---|---|
| Acetic Acid Concentration | 5% | |
| Weight of Collagen | 1 | gr |
| Volume of Glacial acetic acid | 10 | ml |
| Volume of DDW | 190 | ml |
| Final volume of preparation | 200 | ml |

The formulation was mixed for 30 min at 6000 rpm with a lab scale Silverson rotor stator mixer. The mixture was stored overnight in a cooler above the freezing point of the dispersion. With a 25 ml pipette large droplets (<10 mm) of the dispersion were dropped into liquid nitrogen and allowed to freeze. The large droplets were added to a 3 mm stainless steel sieve suspended in a liquid nitrogen bath. The sieve was agitated with a shaker. The droplets, immersed in liquid, nitrogen were ground with a high speed kitchen type mixer. See FIG. 3. Ground particles, smaller than 3 mm fall through the sieve. The ground frozen particles were separated, while immersed in liquid nitrogen, into 3 fractions

(a) 2 to 3 mm

(b) 5 to 2 mm

(c) <0.5 mm

Frozen particle fractions were lyophilized for 5 days at a pressure <60×10⁻³ MBAR. The lyophilized sponges were dehydrothermally cross-linked at 120 C at <1 torr for 3 days.

### EXAMPLE 7

### Wet Versus Dry Sponge Dimensions

Materials and Methods:

A formulation for a collagen dispersion is shown below

For 200 ml of dispersion:

| Collagen Concentration | 5 | mg/ml |
|---|---|---|
| Acetic Acid Concentration | 5% | |
| Weight of Collagen | 1 | gr |
| Volume of Glacial acetic acid | 10 | ml |
| Volume of DDW | 190 | ml |
| Final volume of preparation | 200 | ml |

The formulation was mixed for 30 min at 6000 rpm with a lab scale Silverson rotor stator mixer. The mixture was stored overnight in a cooler above the freezing point of the dispersion. The dispersion was poured into ice cube trays with dimensions of 1″×1″×1.75″. Each tray was filled about ⅔ to ¾ volume. The trays containing the dispersion were placed in a foam polystyrene container with a lid. The whole assembly was placed in a freezer set to −20 C. The intent was to have slow cooling to generate a large pore size. The dispersion was chilled for, at least, 2 days at which point the dispersion is frozen.

Three frozen cubes were quickly removed from the cooler, split in half with a stainless steel knife, and added to a 5 mm stainless steel sieve suspended in a liquid nitrogen bath. The sieve was agitated with a shaker. The cubes, immersed in liquid, nitrogen were ground with a high speed kitchen type mixer. See FIG. 3. Ground particles, smaller than 5 mm fell through the sieve. The ground frozen particles were separated, while immersed in liquid nitrogen, into 3 fractions

(a) 3 to 5 mm

(b) 2 to 3 mm

(c) <2 mm

Frozen particle fractions were lyophilized for 5 days at a pressure <60×10⁻³ MBAR. The lyophilized sponges were dehydrothermally cross-linked at 120 C at <1 torr for 3 days.

Two sets of samples from each of the two largest fractions were imaged, described as Sample 7A and 7B in Table 1 of the overview of the exemplification. Avg maximum particle diameter and avg. particle cross-sectional area were measured with image Pro Plus 4.5, as described in example 5A-1. One set from each of the two largest fractions was wetted in a two step procedure in absolute ethanol. The second set was wetted directly in medium.

The set of four samples were imaged as SEMs. Avg maximum particle diameter and avg. particle area were then measured with Image Pro Plus 4.5, as described in example 5A-1.

| | Millimeters² | Millimeters |
|---|---|---|
| | Avg. Area | Avg. Max. D |
| Sample 7A - Table 1 | | |
| Dry Particles - 3 to 5 mm sieve | 4.35 | 3.03 |
| Same as above wetted directly in medium | 1.86 | 2.05 |
| % Reduction | 57.2 | 32.3 |
| Sample 7A - Table 1 | | |
| Dry Particles - 3 to 5 mm sieve | 4.9 | 3.27 |
| Same as above wetted in 2 step process | 4.91 | 3.02 |
| % Reduction | −0.2 | 7.6 |
| Sample 7B - Table 1 | | |
| Dry Particles - 2 to 3 mm sieve | 2.43 | 2.31 |
| Same as above wetted in 2 step process | 2 | 2.01 |
| % Reduction | 17.7 | 13.0 |
| Sample 7B - Table 1 | | |
| Dry Particles - 2 to 3 mm sieve | 2.53 | 2.28 |
| Same as above wetted directly in medium | 1.29 | 1.75 |
| % Reduction | 49.0 | 23.2 |

Results and Conclusions:
Results are summarized as follows

Shrinkage was excessive for particles wetted directly into medium, where the average particle cross-sectional area is reduced by about 50%. In contrast, there is no shrinkage or minimal shrinkage for particles wetted in the 2 step process, where the values range from ˜0 to 17%.

### EXAMPLE 8

### Cells Cultured on Particulate Sponges

Materials and Methods

Spheres Made in Pentane at −15 C

Spheres from sample no. 4 of example 1B were wetted via the 9 step process described above. They were further washed 3 times with medium prior to being seeded with porcine fibroblasts. About 200 ml of collagen microspheres, stored in D-MEM at 4 C, were transferred to a 500-ml filter apparatus with a 0.2 micron filter. The culture medium was removed by suction and 200 ml of F12/D-MEM medium containing 15% of fetal calf serum, 2 mM glutamine, 1× penicillin/streptomycin, 0.39 mg/ml of L-arginine, 0.19 mg/ml sodium pyruvate, 2 μg/ml of putrescine, 8 μg/ml of insulin and 8 μg/ml of hydrocortisone were added to the drained microspheres. The microspheres were transferred to a sterile 500 ml bottle using a 25 ml pipette.

For study, 9 ml of the washed microspheres were pipetted into a sterile 6-well plate insert, with a diameter of 2.4 cm and a 74 microns mesh at the bottom, in a sterile culture dish with a 10 cm diameter. The cultured medium in each insert was allowed to drain by gravity. Then, the drained microspheres were washed with 10 ml of F12/DMEM and the medium again was drained by gravity. The washing process was repeated one more time, at which time the drained microspheres were transferred to another sterile 6-well plate insert with a diameter of 2.4 cm and a 0.4 micron mesh at the bottom of the insert in a 10 cm diameter sterile culture dish, using a sterile spatula.

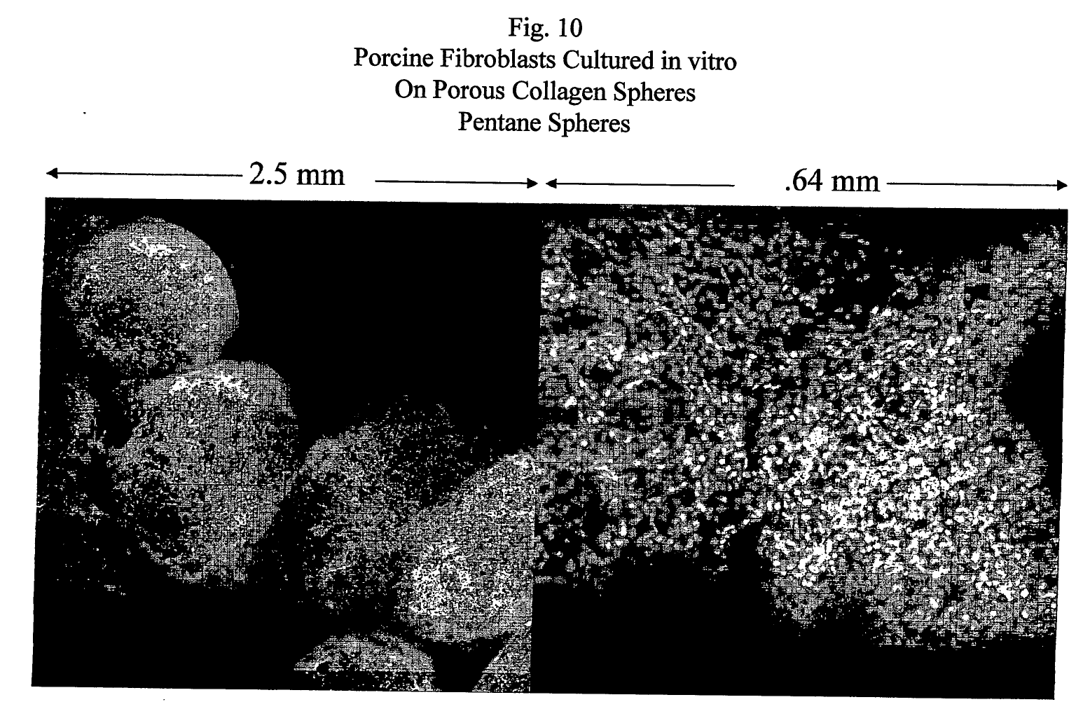

The insert was then placed in a 100 mm sterile petri dish. About 20 ml of the full F12/DMEM medium were added to the dish but not into the insert. Three million fibroblasts in 1 ml of full F12/DMEM medium were added into the insert with the washed and drained microspheres. The dish was then incubated at 37 C in a CO₂ incubator for 2 to 3 hr to facilitate the adsorption of the cells onto the microspheres. After the incubation, more medium was added to the dish until the medium covered the opening of the insert in the dish. The total volume in the dish was about 50 to 60 ml of culture medium. The dish was then incubated at 37 C in a CO₂ incubator for 4 to 6 days. At the time indicated, the microspheres with the cells were pipetted into another 74 micron insert to drain all the culture medium. The microspheres were then washed with 1× phosphate buffered saline in a 6-well plate before they were fixed with 10% formalin for 2 hrs. The microspheres were then washed extensively in the insert, and were subsequently stained and analyzed by confocal microscopy. The confocal photomicrographs are shown in FIG. 10.

Spheres Made in Liquid Nitrogen

Spheres from sample no. 1 of example 1B were used. They were wetted via the 9 step process described above. They were further washed 3 times with medium prior to be seeded with porcine fibroblasts. These were seeded with cells and cultured in vitro as described above in protocol for spheres made in pentane at −15 C

Particle Frozen in Air at −20 C

Particles from no. 7 of example 1B were used. They were wetted via the 9 step process described above. They were further washed 3 times with medium prior to be seeded with porcine fibroblasts. These were seeded with cells and cultured in vitro as described above in protocol for spheres made in pentane at −15 C

### EXAMPLE 9

### Apparatus for Simultaneous Grinding and Sorting

An apparatus for simultaneous grinding and sorting is shown if FIG. 3. Large particles of frozen dispersion are added to the sieve. A high speed mixer is used to reduce particle size. The ground particles are expelled from the sieve as they are reduced to a particle size less than the sieve openings. The vortex created by the grinder facilitates this removal. Agitation of the sieve also promotes removal of the ground particles.

This process permits production of particles with a narrow range of particle sizes in comparison to that produced in a process where grinding and separation are done separately.

### EXAMPLE 10

### Collagen/Chondroitin 6 Sulphate Composites

Materials and Methods:

A collagen dispersion comprising 5 mg/ml collagen and 5% glacial acetic acid was prepared as described above. A solution of 5 mg/ml sodium salt of chondroitin 6 sulphate was also prepared. For the 1^{st} preparation 4 parts of the collagen solution and 1 part of the C6S solution were mixed on a shaker for 15 min. Precipitation occurred. The mixture was poured into ice cube trays. The trays containing the dispersion are placed in a foam polystyrene container with a lid. The whole assembly is placed in a freezer set to −15 C. The intent was to have slow cooling to generate a large pore size. The dispersion is chilled for, at least, 2 days at which point the dispersion is frozen.

Three frozen cubes were quickly removed from the cooler, split in half with a stainless steel knife, and added to a basket constructed of a 3 mm stainless steel sieve. The basket was immersed in liquid nitrogen. While the basket was agitated, the cubes were ground with a high speed mixer. The fractured particles pass through the 3 mm sieve. The resulting particles were then filtered through a 0.5 mm sieve. The particles that remain on the sieve were lyophilized for 5 days at a pressure <60×10⁻³ MBAR. The lyophilized sponges were dehydrothermally cross-linked at 120 C at <1 torr for 3 days.

It should be noted that dehydrothermally cross-linked, collagen sponges (e.g., wetted and dry particulates, e.g., non-spherical) of the invention may comprise a glycosamine glycan. In certain embodiments, as in this example, the glycosamine glycan is chondroitin 6 sulphate.

Results and Conclusions:

Mean pore area and mean max. pore diameter were measured with Image Pro Plus 4.5 as described above in example 1E.

| | | | | | | | Mean | Mean |
|---|---|---|---|---|---|---|---|---|
| | | | Collagen/C6S | | | | Area | Max. Diam. |
| No | Coolant | Temp C. | Mg/ml | Size | Acid % | Mag x | Microns² | microns |
| 44-2 | air | −20 | 4/1 (C/C6S) pre | | 5 | 200 | 867 | 22 |
| 45-1 | air | −20 | 4/1 (C/C6S) tray | | 5 | 200 | 578 | 17.8 |
| 45-3 | air | −20 | 4/1 (C/C6S) tray | | 5 | 60 | .7098 | 63 |

### EXAMPLE 11

### Volume Calculations for Sponges

Calculation of Volume and Change in Volume for Non-Spherical Particles

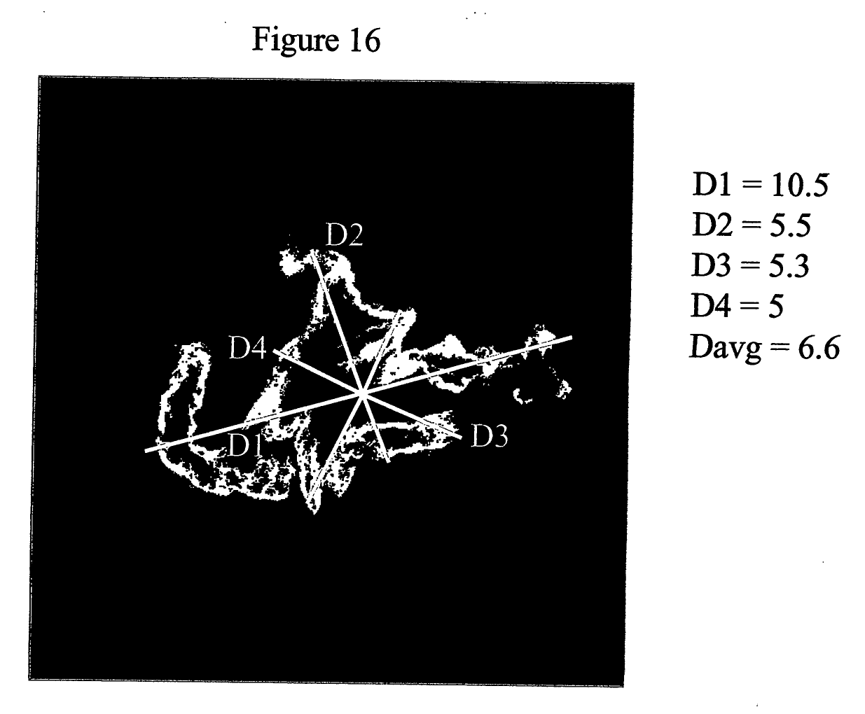

Using any of the methods described herein, a photomicrograph of a particle or a population of particles, e.g., wet or dry, is produced. For each particle image:
(a) The longest possible straight line is drawn through the two-dimensional image and the length of the line intersecting the two extremities of the wetted sphere is measured. This length is designated as D1.
(b) A straight line is then drawn perpendicular to D1 line through the center point of D1, and the length of the line intersecting the 2 extremities of the wetted sphere is measured. This length is designated as D2.
(c) Two more lines are drawn through the center point of D1 that intersect each other at 90 degrees and intersect D1 at 45 degrees. The length of the two lines intersecting the four extremities of the wetted sphere is then measured. These lengths are designated as D3 and D4
(d) The average value of D for each particle is calculated using the following equation:
   D_{avg}=(D1+D2+D3+D4)/4.
(e) The average value of the diameter of the particles in the population is then calculated using the following formula:
   D_{avg/pop}=(D_{avg}1+D_{avg}2+ . . . . +D_{avg}N)/N
where N=the number of particles in the image
(f) The average particle radius is then calculated using the following formula:
   Rns=(Davg/pop)/2
(g) The average particle volume is then calculated using the following formula:
   Vns=(4/3)*(π)*Rₙₛ³
   FIG. 16 provides an illustration of the line placement and line measurement.

To evaluate the percentage change in volume for a dry versus a wet particulate, the average particle volume (Vns) for a population of dry particles was measured. The same population of dry particles was wetted. The average particle volume of the wetted particulates was then measured. The percentage change in average particle volume (APV %) was then calculated using the following formula:
APV%=100×(Average particle volume Vns Dry−Average particle volume Vns Wet)/Average particle volume Vns Dry
Calculation of Volume and Change in Volume of Spherical Particles

Using any of the methods described here in, a photomicrograph of a particle or a population of particles, e.g., Wet or dry, is produced. This procedure is for spherical particles or approximately spherical particles. For each particle image:
(a) The longest possible straight line is drawn through the two-dimensional image and the length of this line is measured. This length is designated as the diameter Dᵢ
(b) The particle radius is then calculated by using the following equation:
   Rᵢ=Dᵢ/2
(c) The average radius of the particles in a population is calculated using the following equation:
   Rₛ=(R1+R2+ . . . Rₙ)/n
(d) The average particle volume is then calculated using the following equation:
   V_{S}=(4/3)*(π)*Rₛ³

To evaluate the percentage change in volume for a dry versus a wet particulate, the average particle volume (Vns) for a population of dry particles was measured. The same population of dry particles was wetted. The average particle volume of the wetted particulates was then measured. The percentage change in average particle volume (APV %) was then calculated using the following formula:
APV%=100×(Average particle volume Vns Dry−Average particle volume Vns Wet)/Average particle volume Vns Dry

### EXAMPLE 12

### Small Particles by Spraying into a Chilling Bath

Small particulate collagen sponges may be prepared by one of the three following methods.
1. Small particulate collagen sponges may be prepared by
   atomizing a dispersion of insoluble collagen or a solution of soluble collagen into a cryogenic bath by metering the dispersion or solution of collagen through a nozzle that is immersed in the cryogenic bath.
   Lyophilizing the frozen particles,
2. Small particulate collagen sponges may be prepared by
   atomizing a dispersion of insoluble collagen or a solution of soluble collagen into a cryogenic bath by metering the dispersion or solution of collagen through a nozzle that is immersed in the cryogenic bath.
   Lyophilizing the frozen particles
   Cross-linking
3. Small particulate collagen sponges wetted in an aqueous medium may be prepared by
   atomizing a dispersion of insoluble collagen or a solution of soluble collagen into a cryogenic bath by metering the dispersion or solution of collagen through a nozzle that is immersed in the cryogenic bath.
   Lyophilizing the frozen particles
   Cross-linking
   wetting the dehydrothermally cross-linked sponges in a non-aqueous water soluble solvent at reduced pressure, resulting in dehydrothermally cross-linked sponges wetted with a non-aqueous medium
   exposing the wetted, dehydrothermally cross-linked sponges to a gradient of solvent mixtures comprising the non-aqueous solvent and water, starting with a high concentration of the non-aqueous solvent and ending with water or an aqueous solution to form a dehydrothermally cross-linked sponges wetted with an aqueous medium. Alternatively, wash directly in an aqueous medium

### EXAMPLE 13

### Fast Multiple Step Wetting Process

Dry collagen particles or spheres were added to a container comprising ethanol (absolute) and then transferred to a bell shape vacuum desiccator. Vacuum was then applied for 5 min to release all the air bubbles trapped in the pores, and the collagen particles sank to the bottom of the container. The ethanol wetted collagen particles or spheres were transferred to a filter unit (0.2 micron).

The ethanol was then removed by filtration to a point where the wetted particulates were packed without a visible layer of ethanol on top of the packed particles. 50% ethanol/50% phosphate buffer solution (PBS) was added to the filter unit (using 70% EtOH/30% PBS a fine white precipitate forms in the solution). The particles or spheres were allowed to equilibrate with the ethanol/PBS mixture for about 10 min. The ethanol/PBS mixture was removed via filtration to a point where the wetted particulates were packed, but without a visible layer of ethanol/PBS mixture, on top of the packed spheres.

The processes of washing and filtering was then repeated with 100% PBS and then with 1×DMEM. After removing the 1×DMEM by suction, 2× the volume of the packed volume of the particles or spheres of 1×DMEM containing 10% fetal calf serum and penicilin and streptomycin were added. The suspension was stirred and allowed to equilibrate for 10 min. The suspension was then transferred to a sterile bottle and stored at 4 C for at least one to two days. Before use, the microspheres suspension was transferred into a filter apparatus (0.2 micron) and washed once, as described previously, with 1×DMEM containing 10% fetal calf serum and penicillin and streptomycin.

After removing the medium by filtration, 2× volume of the packed particles or spheres of the same culture medium were added. The particles or spheres suspension is transferred to a sterile bottle and was ready to be used. The wetted particles or spheres were kept at 4 C.

Alternatively, after washing the particles or spheres with 1×DMEM, the washing process can be repeated twice with 1×DMEM containing 10% fetal calf serum and penicilin and streptomycin. After the final wash, 2× the volume of the packed volume of the particles or spheres of 1×DMEM containing 10% fetal calf serum and penicillin and streptomycin are added. The particles or spheres suspension is then transferred to a sterile bottle and is ready to be used. Again, the wetted particles or spheres are kept at 4 C.

### EXAMPLE 14

### Fast 2 Step Wetting Process

Dry collagen particles or spheres were added to a container comprising ethanol (absolute) and then transferred to a bell shape vacuum desiccator. Vacuum was then applied for 5 min to release all the air bubbles trapped in the pores, and the collagen particles sank to the bottom of the container. The ethanol wetted collagen particles or spheres were transferred to a filter unit (0.2 micron).

The ethanol was then removed by filtration to a point where the wetted particulates were packed without a visible layer of ethanol on top of the packed particles. Water or PBS (phosphate buffer solution) was added to the filter unit. The particles or spheres were allowed to equilibrate for about 10 min. The water or PBS was then removed via filtration to a point where the wetted particulates are packed but without a visible layer of liquid on top of the packed spheres. The processes of washing and filtering was repeated with water or PBS. The spheres were then wetted with DMEM as described in 13.

### EXAMPLE 15

### Compositions and Processes Comprising Hydroxy Apatite

A mixture comprising 1 mg/ml to 10 mg/ml of collagen and hydroxyapatite in 1% to 10% glacial acetic acid is prepared, wherein the minimum percentage of collagen in the collagen+hydroxy apatite mixture is 5%. The mixture is poured into ice cube trays. The trays containing the dispersion are then placed in a foam polystyrene container with a lid. The whole assembly is placed in a freezer set to −15 C. The assembly was slow cooled to generate a large pore size. The dispersion was chilled for at least 2 days, at which point the dispersion is frozen.

Three frozen cubes are quickly removed from the cooler, split in half with a stainless steel knife, and added to a basket constructed of a 3 mm stainless steel sieve. The basket is immersed in liquid nitrogen. While the basket is agitated, the cubes are ground with a high speed mixer. The fractured particles pass through the 3 mm sieve. The resulting particles are then filtered through a 0.5 mm sieve. The particles that remain on the sieve are lyophilized for 5 days at a pressure <60×10⁻³ MBAR. The lyophilized sponges are dehydrothermally cross-linked at 120 C at <1 torr for 3 days.

The dry particles are wetted by stepwise wetting procedures already described for other particulates.

Compositions incorporating hydroxy apatite, a significant component of the extrcellular matrix in bone (collagen being another major component of the extracellular matrix in bone), are useful alone, or in composites, as implantable bone tissue supplements.

### EXAMPLE 16

### Islets—Microspheres Comprising Cells with Shell of Complex Coacervate

Spheres made in Pentane at −15 C Spheres from Sample no. 4 of example 1B are wetted via the 9 step process described above. They are further washed 3 times with medium, prior to being seeded with porcine fibroblasts. About 200 ml of collagen microspheres, stored in D-MEM at 4 C, are transferred to a 500-ml filter apparatus with a 0.2 micron filter. The culture medium is removed by suction and 200 ml of F12/D-MEM medium containing 15% of fetal calf serum, 2 mM glutamine, 1× penicillin/streptomycin, 0.39 mg/ml of L-arginine, 0.19 mg/ml sodium pyruvate, 2 μg/ml of putrescine, 8 μg/ml of insulin and 8 μg/ml of hydrocortisone are added to the drained microspheres. The microspheres are transferred to a sterile 500 ml bottle using a 25 ml pipette.

For study, 9 ml of the washed microspheres are pipetted into a sterile 6-well plate insert, with a diameter of 2.4 cm and a 74 microns mesh at the bottom, in a sterile culture dish with a 10 cm diameter. The cultured medium in each insert is allowed to drain by gravity. Then, the drained microspheres are washed with 10 ml of F12/DMEM and the medium again was drained by gravity. The washing process is repeated one more time. Then, the drained microspheres are transferred to another sterile 6-well plate insert with a diameter of 2.4 cm and a 0.4 micron mesh at the bottom of the insert in a 10 cm diameter sterile culture dish, using a sterile spatula. The insert is then placed in a 100 mm sterile Petri dish. About 20 ml of the full F12/DMEM medium are added to the dish but not into the insert. Three million fibroblasts in 1 ml of full F12/DMEM medium are added into the insert with the washed and drained microspheres. The dish is then incubated at 37 C in a CO₂ incubator for 2 to 3 hr to facilitate the adsorption of the cells onto the microspheres. After the incubation, more medium is added to the dish until the medium covered the opening of the insert in the dish. The total volume in the dish is about 50 to 60 ml of culture medium. The dish is then incubated at 37 C in a CO₂ incubator for 4 to 6 days.

The calcium level is adjusted and the microspheres comprising cells are incubated. The microspheres comprising cells are added to an alginate solution. Upon addition a complex coacervate shell forms around the microspheres comprising cells

### EXAMPLE 17A

### Continuous Process for the Manufacture of Dermal Membrane

(a) Preparation of Particulate Collagen Dispersion

About 200 mL of dehydrothermally cross-linked dry collagen particulates prepared according to the methods of the invention were suspended in 200 mL of absolute ethanol, in a sterile 500-mL conical flask with a screw cap. The suspension was subjected to a vacuum to remove air in the particulates.

After the particulates sank to the bottom of the flask, the liquid was removed by first decanting, followed by using a pipette. About 200 mL of 70% ethanol in PBS was added to the flask, which was then shaken with a wrist shaker to mix the suspension until all the particulates sank to the bottom of the flask. The liquid was subsequently removed as previously described.

About 200 mL of 50% ethanol in PBS were then added, the suspension was shaken, and the liquid was removed after the particulates sank to the bottom of the flask. The process was repeated, continuing with 30% ethanol in PBS, 100% PBS.

Finally D-MEM containing 10% fetal calf serum supplemented with glutamine and penicillin/streptomycin was added to the particulates. The particulates were stored in D-MEM at 4° C.

(b) Preparation of Cell Dispersion in a Gellable Collagen Solution

Acid soluble collagen solution (0.35 mL), containing 1×D-MEM and 10% fetal calf serum at 4° C., was mixed with D-MEM (0.2 mL) containing 10% fetal calf serum and (1×10⁵) normal human fibroblasts at 4° C.

(c) Preparation of Tissue Composite Using Collagen Particulates

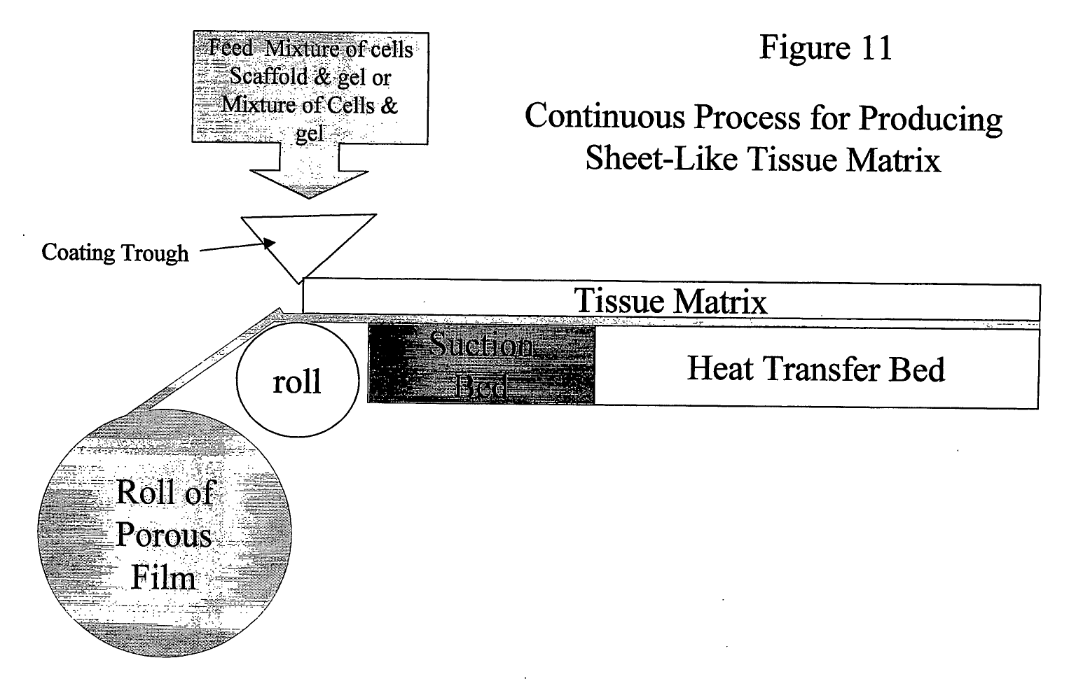

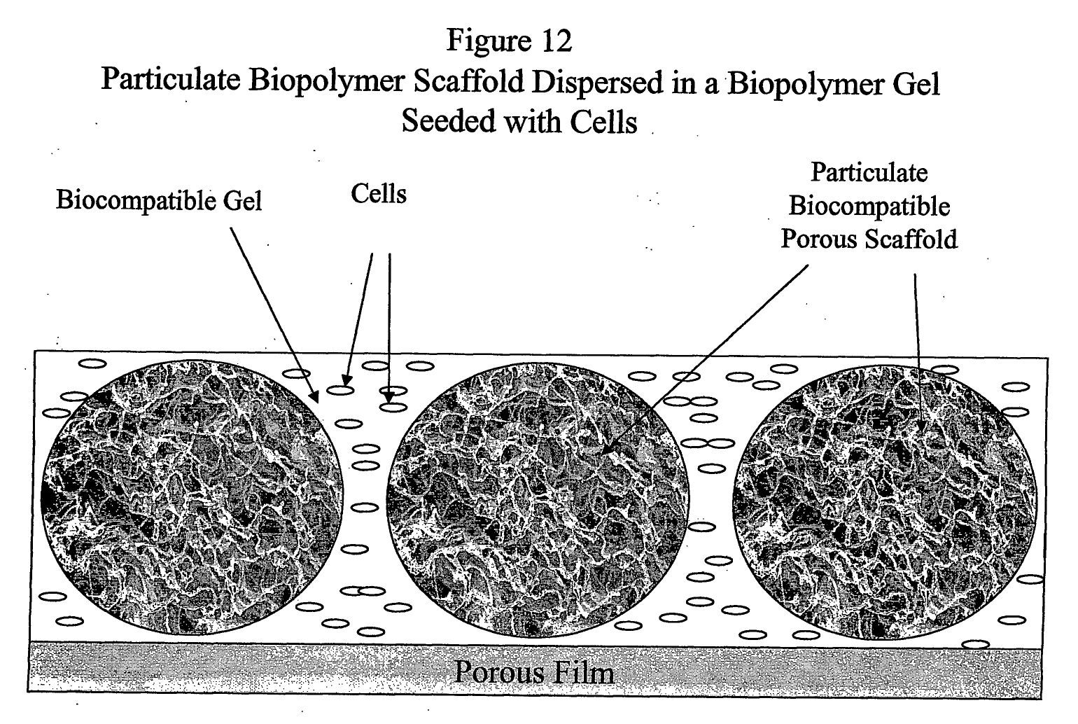

The particulate collagen dispersion of (a) and the cell dispersion of (b) are mixed in a ratio of 1.5/0.45 to 3/0.45 while maintained at a temperature of 4 C. The mixture is added to the coating trough of the apparatus shown in FIG. 11. The mixture is coated onto the moving polymer film. Excess culture medium is optionally removed via suction through the porous film by the suction bed as shown below while still maintaining the temperature at ˜4 C. The coated film is then heated to 37 C by the heat transfer bed and gellation of the collagen solution occurs. A schematic of the tissue matrix generated in using this process is shown in the FIG. 12. The suction bed is flat plate, e.g., steel, comprising small holes. A vacuum is applied through the holes causing excess culture medium to be sucked from dispersion through porous polymer film and away from the tissue composite. A heat transfer bed is a plate, e.g., a steel plate, heated to about 37 C, and is positioned to be in contact with the polymer film side of the tissue matrix

The sheet-like composite may be cut into the shape desired for use. It is stored in culture medium until application.

### EXAMPLE 17B

### Continuous Process for the Manufacture of Dermal Membrane

A non-spherical particulate collagen particle is prepared in accordance with the processes of the invention. An aqueous dispersion of the particles is prepared as described in Example 17A part (a) above. The particle dispersion is mixed with a cell dispersion. The volume of cell culture medium is maintained at a level just greater than that required to wet the ingredients. The mixture is maintained in a quiescent state to allow the cells to attach. Additional medium is added and cells are culture in a bioreactor to the desired density.

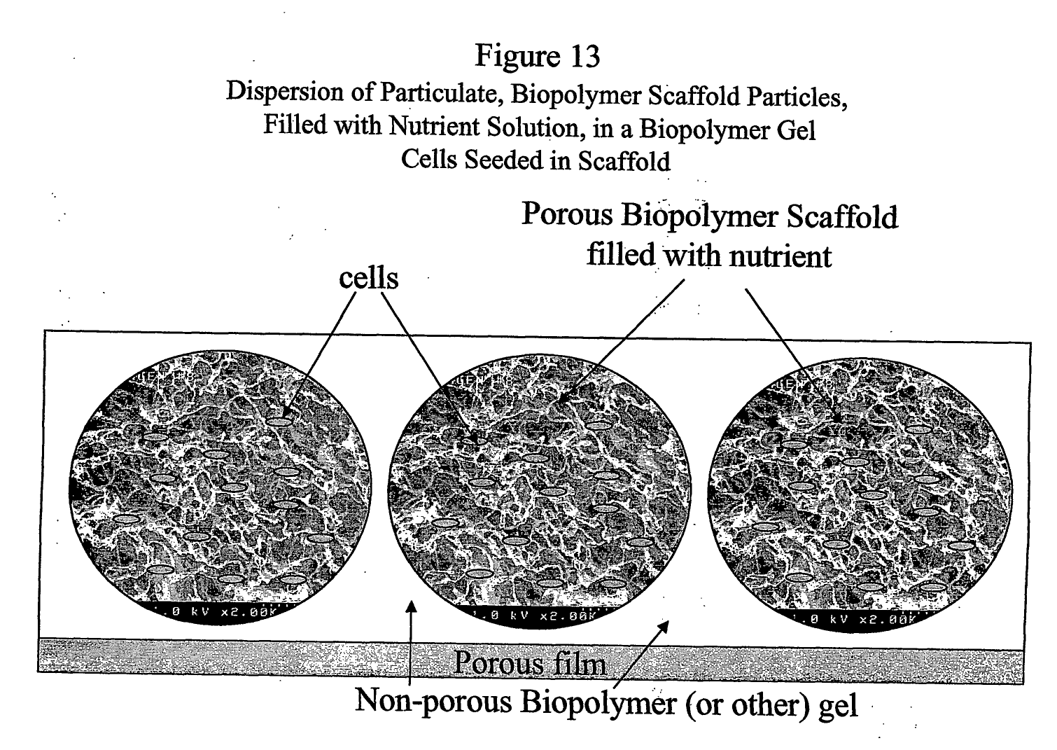

The dispersion particulate collagen with attached cells is mixed with a gellable collagen solution and the temperature maintained at ˜4 C. The mixture is added to the coating trough of the apparatus shown in FIG. 11. An engineered tissue composite is produced in a similar manner as that described in Example 17A part (c). A schematic of the composite is shown in the FIG. 13.

### EXAMPLE 17C

### Continuous Process for the Manufacture of Dermal Membrane

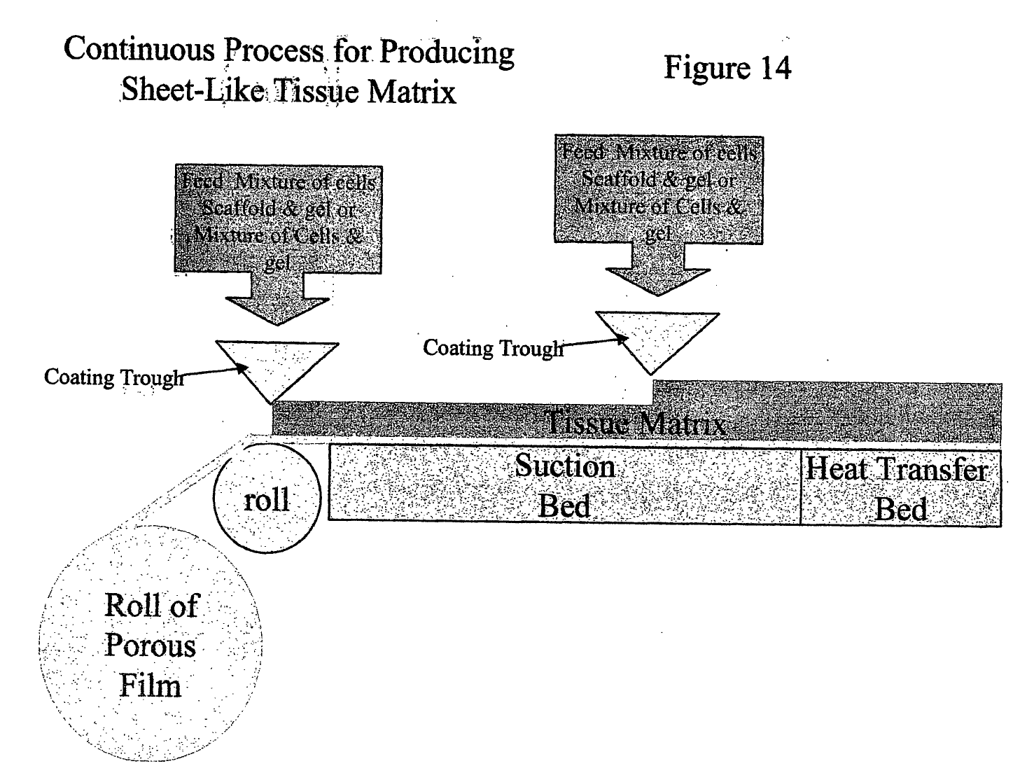

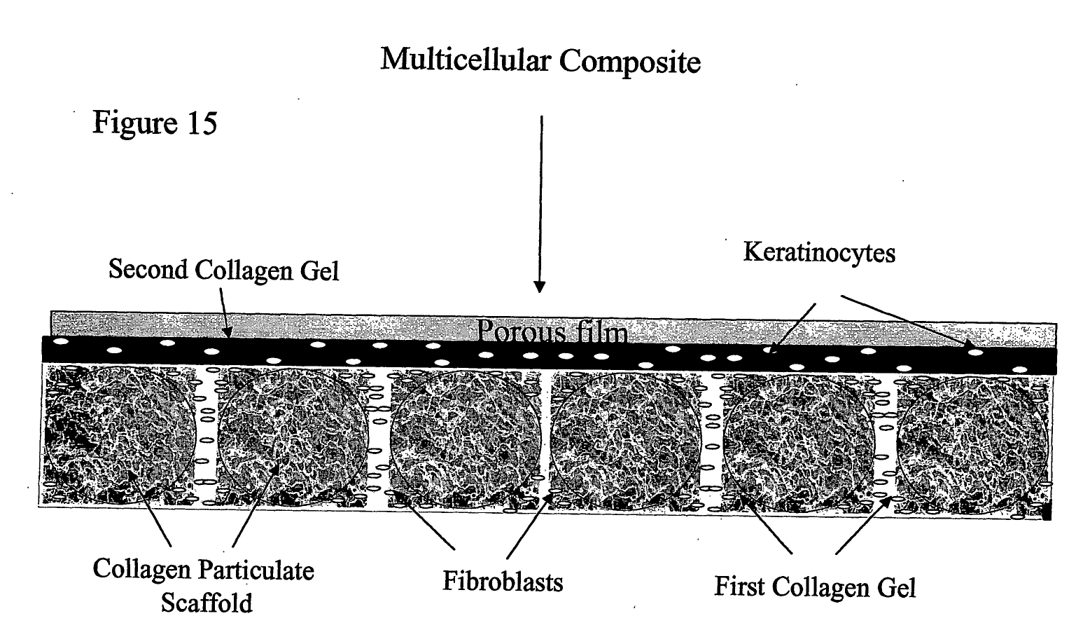

This is an example of a process to produce a two layer tissue matrix using the apparatus depicted in FIG. 14. The apparatus is similar to that shown in FIG. 11 with the exception that it contains two coating stations. The second coating station is used to coat a dispersion of cells in a gellable collagen solution to the composite (e.g., as would be produced by the apparatus described in example 17A) to form the composite shown in FIG. 15.

A dispersion of cells in a gellable collagen solution is coated at the 1^{st} coating station and temperature is maintained below the gelling temperature. A dispersion of particulate collagen and cells in a gellable collagen solution is coated at the 2^{nd} coating station while the temperature is maintained below the gel temperature. Optionally, excess nutrient medium is removed through the porous film via the suction bed. The bi-layer tissue matrix heated to the gel temperature on the heat transfer bed to gel the composite.

### Equivalents

Those skilled in the art will recognize, or be able to ascertain using no more than routine experimentation, many equivalents to the specific embodiments and methods described herein. Such equivalents are intended to be encompassed by the scope of the following claims.

### INCORPORATION BY REFERENCE

The entire contents of all patents, published patent applications, and referenced figures and other references cited herein are hereby expressly incorporated herein in their entireties by reference.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. A dehydrothermally cross-linked, collagen sponge wetted with an aqueous medium prepared by a method comprising:
(a) preparing an aqueous dispersion of insoluble collagen or solution of soluble collagen;
(b) casting the dispersion or the solution of soluble collagen into a shape desired for end use;
(c) freezing the cast shape;
(d) lyophilizing the frozen, cast shape to form a collagen sponge;
(e) dehydrothermally cross-linking the lyophilized collagen sponge;
(f) wetting the dehydrothermally cross-linked sponge in a non-aqueous water soluble solvent; and
(g) washing the sponge wetted with a non-aqueous water soluble solvent with an aqueous solution.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. The collagen sponge of claim 15 wherein, in said method, the average cross-sectional area of a plurality of said sponges when wet is within +/−20% of the value for average cross-sectional area of said sponges when unwetted.

88. The collagen sponge of claim 15 wherein the average maximum diameter of a plurality of said sponges is within +/−20% of the value for the average maximum diameter of said plurality of said sponges when unwetted.

89. The collagen sponge of claim 15 wherein said sponge is a particulate.

90. The collagen sponge of claim 15 wherein said sponge is spherical.

91. The collagen sponge of claim 15 wherein said sponge is non-spherical.

92. The collagen sponge of claim 15 wherein said sponge has a sheet form.

93. The collagen sponge of claim 89 wherein said sponge has a diameter no less than 0.25 mm and no greater than 10 mm.

94. The collagen sponge of claim 15 wherein, in said method, collagen is present in said dispersion in an amount no less than 0.05% and no greater than 10% based on weight of said dispersion.

95. The collagen sponge of claim 15 wherein, in said method, said dispersion or solution further comprises a protein, carbohydrate, lipid, or mixture thereof.

96. The collagen sponge of claim 15 wherein said sponge has an average maximum pore diameter no less than 3μ and no greater than 16μ.

97. The collagen sponge of claim 15 wherein said sponge has an average maximum pore area no less than 10 mm² and no greater than 200 mm².

98. The collagen sponge of claim 15 wherein step (g) comprises washing with a series of mixtures comprising a non-aqueous water soluble solvent and water, wherein progressively higher levels of water are employed from one wash to the next wash.

99. The collagen sponge of claim 15 wherein the lyophilized collagen sponge is subjected to milling in a cryogenic medium before dehydrothermally cross-linking the lyophilized collagen sponge.

100. The collagen sponge of claim 99 wherein the milled particles are separated into ranges by sieving in a cryogenic medium after milling and before lyophilization.

101. The collagen sponge of claim 99 wherein the lyophilized collagen sponge is subjected to simultaneously milling and sieving the shape into particles in a cryogenic medium prior to dehydrothermally cross-linking the lyophilized collagen sponge.

102. The collagen sponge of claim 15 wherein step (b) involves casting a shape by employing a mold.

103. The collagen sponge of claim 15 wherein said freezing of said cast shape comprises the use of liquid nitrogen.

104. A composition comprising a microorganism and the collagen sponge of claim 15.

105. A composition comprising a pharmaceutical agent and the collagen sponge of claim 15.
